(19) United States
(12) Patent Application Publication
(10) Pub. No.: US 2023/0225750 A1
(43) Pub. Date: Jul. 20, 2023

(54) INTRANASAL BALLOON COMPRESSION FOR TREATMENT OF CHRONIC RHINITIS

(71) Applicant: Acclarent, Inc., Irvine, CA (US)

(72) Inventors: Jetmir Palushi, Irvine, CA (US); Fatemeh Akbarian, Rancho Palos Verdes, CA (US); Athanasios Papadakis, Newport Beach, CA (US); Ehsan Shameli, Irvine, CA (US); Marc Dean, Fort Worth, TX (US); William J. Kane, Newport Coast, CA (US); Henry F. Salazar, Pico Rivera, CA (US); Julie M. Taylor, Yorba Linda, CA (US); Cesar Fuentes-Ortega, Pasadena, CA (US); Shubhayu Basu, Anaheim, CA (US)

(21) Appl. No.: 18/120,027

(22) Filed: Mar. 10, 2023

Related U.S. Application Data
(63) Continuation-in-part of application No. 16/396,846, filed on Apr. 29, 2019.
(60) Provisional application No. 62/674,767, filed on May 22, 2018.
(60) Provisional application No. 63/329,044, filed on Apr. 8, 2022.

(51) Int. Cl. A61B 17/24; A61M 29/02; A61B 1/233

(52) U.S. Cl. CPC A61B 17/24; A61M 29/02; A61B 1/233; A61M 2210/0681

(57) ABSTRACT

A method includes inserting a dilation catheter into a nostril of a patient and positioning a first dilator of the dilation catheter between a turbinate of the patient and an adjacent lateral nasal wall of the patient. The method also includes expanding the first dilator, thereby applying pressure to the turbinate of the patient, and removing the dilation catheter from the nostril of the patient.

### PRIORITY

This application is a continuation-in-part of U.S. patent application Ser. No. 16/396,846, entitled “Method of Treating Deviated Nasal Septum, Enlarged Nasal Turbinate, or Mucosal Hypertrophy,” filed Apr. 29, 2019, the disclosure of which is incorporated by reference herein, in its entirety. U.S. patent application Ser. No. 16/396,846 claims priority to U.S. Provisional Pat. App. No. 62/674,767, entitled “Method of Treating Deviated Nasal Septum, Enlarged Nasal Turbinate, or Mucosal Hypertrophy,” filed May 22, 2018, the disclosure of which is incorporated by reference herein, in its entirety.

This application also claims priority to U.S. Provisional Pat. App. No. 63/329,044, entitled “Intranasal Balloon Compression for Treatment of Chronic Rhinitis,” filed Apr. 8, 2022, the disclosure of which is incorporated by reference herein, in its entirety.

### BACKGROUND

A human nasal cavity includes a nasal septum and a set of turbinates. A turbinate (or nasal conchae) is a long, narrow and curled bone shelf which protrudes medially into the nasal passages. Turbinates divide the nasal airway into three (or in some cases four) groove-like air passages (i.e., nasi meatae) and are responsible for forcing inhaled air to flow in a steady, regular pattern around the largest possible surface of cilia, and climate controlling tissue of the nasal passage. Turbinates are composed of pseudo-stratified columnar ciliated respiratory epithelium with a thick, vascular and erectile glandular tissue layer. The turbinates are located laterally in the nasal cavities, curling medially and downwardly into the nasal airway. In many cases, there are three pairs of turbinates—superior turbinates, middle turbinates, and inferior turbinates. In some cases, there is an additional pair of turbinates known as the supreme turbinates. Each turbinate pair is composed of one turbinate in either side of the nasal cavity, divided by the nasal septum.

The nasal septum is formed of bone and cartilage, with an exterior lining of mucosal tissue. When the cartilage or bone is off-center (i.e., deviated laterally) or crooked, the condition may be referred to as a deviated septum. A deviated septum may come into close proximity to an adjacent turbinate, or even engage an adjacent turbinate, and thereby create a restriction or blockage in the nasal passageway, which may lead to breathing difficulties, bleeding, pain, and/or other undesirable conditions in a patient. It may therefore be desirable to treat a deviated septum to ameliorate and prevent such undesirable conditions.

Some conventional approaches to addressing a deviated nasal septum may include a septoplasty procedure. A septoplasty procedure may include making an incision in the mucosal tissue of the nasal septum, removing at least a portion of the nasal septum, straightening the removed nasal septum, and then inserting the straightened nasal septum into the mucosal tissue. Such an approach may be considered aggressive and time consuming. It may be desirable to address a deviated nasal septum in a manner that is less invasive than a conventional septoplasty procedure, under local anesthesia in a doctor's office. It may also be desirable to address a deviated nasal septum in a manner that does not require the complexity and skill associated with a septoplasty procedure.

Some patients may also suffer from a turbinate that has become enlarged due to inflammation or infection. Like a deviated nasal septum, an enlarged turbinate may lead to breathing difficulties, bleeding, pain, and/or other undesirable conditions in a patient. Some conventional approaches to addressing an enlarged turbinate may include reducing the turbinate by using scissors to cut the turbinate, using forceps to crush the turbinate, or using energy to desiccate the turbinate. It may be desirable to address an enlarged turbinate using less invasive methods that require less complexity and skill than the turbinate reduction procedures noted above.

Some patients may also suffer from a hypertrophy of mucosal tissue in the nasal cavity. In some instances, the collapsed mucosal tissue may obstruct air flowing through the nasal cavity. Some conventional approaches to addressing collapsed mucosal tissue may include resecting the collapsed mucosal tissue to provide a clear passage air flow through the nasal cavity. It may be desirable to address collapsed mucosal tissue in a nasal cavity using less invasive methods that require less complexity and skill than the mucosa resection procedures noted above.

Rhinitis is a medical condition that presents as irritation and inflammation of the mucous membrane within the nasal cavity. The inflammation results in the generation of excessive amounts of mucus, which can cause runny nose, nasal congestion, sneezing, and/or post-nasal drip. Allergenic rhinitis is an allergic reaction to environmental factors such as airborne allergens, while non-allergenic (or “vasomotor”) rhinitis is a chronic condition that presents independently of environmental factors. Conventional treatments for rhinitis include antihistamines, topical or systemic corticosteroids, and topical anticholinergics, for example.

For cases of intractable rhinitis in which the symptoms are severe and persistent, an additional treatment option is the surgical removal of a portion of the vidian (or “pterygoid”) nerve—a procedure known as vidian neurectomy. The theoretical basis for vidian neurectomy is that rhinitis is caused by an imbalance between parasympathetic and sympathetic innervation of the nasal cavity, and the resultant over stimulation of mucous glands of the mucous membrane. Vidian neurectomy aims to disrupt this imbalance and reduce nasal mucus secretions via surgical treatment of the vidian nerve. However, in some instances, vidian neurectomy can cause collateral damage to the lacrimal gland, which is innervated by the vidian nerve. Such damage to the lacrimal gland may result in long-term health complications for the patient, such as chronic dry eye. Posterior nasal neurectomy, or surgical removal of a portion of the posterior nasal nerves, may be an effective alternative to vidian neurectomy for treating intractable rhinitis.

While several systems and methods have been made and used to treat a deviated nasal septum and other anatomical structures within the nasal cavity, and while instruments and methods for performing vidian neurectomies and posterior nasal neurectomies are known, it is believed that no one prior to the inventors has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements and in which:

FIG. 1A depicts a side schematic view of an exemplary dilation catheter, with a dilator of the dilation catheter in a non-expanded state;

FIG. 1B depicts a side schematic view of the dilation catheter of FIG. 1A, with the dilator in an expanded state;

FIG. 2A depicts a schematic view, along a coronal plane, of anatomical structures associated with a nasal cavity of a patient, including a nasal septum in a deviated state, before a first exemplary treatment procedure;

FIG. 2B depicts a schematic view, along a coronal plane, of the anatomical structures associated with the nasal cavity of the patient of FIG. 2A, with a distal portion of the dilation catheter of FIG. 1A inserted through a nostril of the patient, and with the dilator of the dilation catheter in the non-expanded state;

FIG. 2C depicts a schematic view, along a coronal plane, of the anatomical structures associated with the nasal cavity of the patient of FIG. 2A, with the distal portion of the dilation catheter of FIG. 1A inserted through a nostril of the patient, and with the dilator of the dilation catheter in the expanded state;

FIG. 2D depicts a schematic view, along a coronal plane, of the anatomical structures associated with the nasal cavity of the patient of FIG. 2A, with the dilation catheter of FIG. 1A removed from the patient, and with the nasal septum in a non-deviated state;

FIG. 3A depicts a schematic view, along an axial plane, of anatomical structures associated with a nasal cavity of a patient, including the nasal septum in the deviated state of FIG. 2A, before the first exemplary treatment procedure;

FIG. 3B depicts a schematic view, along an axial plane, of the anatomical structures associated with the nasal cavity of the patient of FIG. 3A, with the distal portion of the dilation catheter of FIG. 1A inserted through a nostril of the patient, and with the dilator of the dilation catheter in the non-expanded state;

FIG. 3C depicts a schematic view, along an axial plane, of the anatomical structures associated with the nasal cavity of the patient of FIG. 3A, with the distal portion of the dilation catheter of FIG. 1A inserted through a nostril of the patient, and with the dilator of the dilation catheter in the expanded state;

FIG. 3D depicts a schematic view, along an axial plane, of the anatomical structures associated with the nasal cavity of the patient of FIG. 3A, with the dilation catheter of FIG. 1A removed from the patient, and with the nasal septum in a non-deviated state;

FIG. 4A depicts a schematic view, along a coronal plane, of anatomical structures associated with a nasal cavity of a patient, including a nasal septum in a deviated state, before a second exemplary treatment procedure;

FIG. 4B depicts a schematic view, along a coronal plane, of the anatomical structures associated with the nasal cavity of the patient of FIG. 4A, with distal portions of two dilation catheters inserted through respective nostrils of the patient, and with the dilator of each dilation catheter in the non-expanded state;

FIG. 4C depicts a schematic view, along a coronal plane, of the anatomical structures associated with the nasal cavity of the patient of FIG. 4A, with distal portions of two dilation catheters inserted through respective nostrils of the patient, and with the dilator of each dilation catheter in the expanded state;

FIG. 4D depicts a schematic view, along a coronal plane, of the anatomical structures associated with the nasal cavity of the patient of FIG. 4A, with the dilation catheters removed from the patient, and with the nasal septum in a non-deviated state;

FIG. 5A depicts a schematic view, along an axial plane, of anatomical structures associated with a nasal cavity of a patient, including the nasal septum in the deviated state of FIG. 4A, before the second exemplary treatment procedure;

FIG. 5B depicts a schematic view, along an axial plane, of the anatomical structures associated with the nasal cavity of the patient of FIG. 5A, with distal portions of the two dilation catheters inserted through respective nostrils of the patient, and with the dilator of each dilation catheter in the non-expanded state;

FIG. 5C depicts a schematic view, along an axial plane, of the anatomical structures associated with the nasal cavity of the patient of FIG. 5A, with distal portions of the two dilation catheters inserted through respective nostrils of the patient, and with the dilator of each dilation catheter in the expanded state;

FIG. 5D depicts a schematic view, along an axial plane, of the anatomical structures associated with the nasal cavity of the patient of FIG. 5A, with the dilation catheters removed from the patient, and with the nasal septum in a non-deviated state;

FIG. 6 depicts a schematic view, along a coronal plane, of the anatomical structures associated with the nasal cavity of the patient of FIG. 5A, with distal portions of two alternative dilation catheters inserted through respective nostrils of the patient, and with the dilator of each dilation catheter in the expanded state at different respective outer diameters;

FIG. 7 depicts a schematic view, along a coronal plane, of the anatomical structures associated with the nasal cavity of the patient of FIG. 5A, with distal portions of two alternative dilation catheters inserted through respective nostrils of the patient, with the dilator of each dilation catheter in the expanded state, and with one dilator being pushed to a higher vertical position than the other dilator;

FIG. 8A depicts a schematic view, along a coronal plane, of anatomical structures associated with a nasal cavity of a patient, with a first dilation catheter positioned between an inferior turbinate and the nasal septum, with a second dilation catheter positioned between the inferior turbinate and the lateral nasal wall, and with dilators of both dilation catheters in the non-expanded state;

FIG. 8B depicts a schematic view, along a coronal plane, of anatomical structures associated with a nasal cavity of a patient of FIG. 8A, with the first dilation catheter positioned between the inferior turbinate and the nasal septum, with the second dilation catheter positioned between the inferior turbinate and the lateral nasal wall, and with dilators of both dilation catheters in the expanded state;

FIG. 9A depicts a schematic view, along an axial plane, of the anatomical structures associated with the nasal cavity of the patient of FIG. 8A, with the first dilation catheter positioned between the inferior turbinate and the nasal septum, with the second dilation catheter positioned between the inferior turbinate and the lateral nasal wall, and with dilators of both dilation catheters in the non-expanded state;

FIG. 9B depicts a schematic view, along an axial plane, of the anatomical structures associated with the nasal cavity of the patient of FIG. 8A, with the first dilation catheter positioned between the inferior turbinate and the nasal septum, with the second dilation catheter positioned between the inferior turbinate and the lateral nasal wall, and with dilators of both dilation catheters in the expanded state;

FIG. 10 depicts a side plan view of an exemplary dilation catheter, with a dilator of the dilation catheter in an expanded state;

FIG. 11 depicts a side plan view of an exemplary dilation catheter, with a dilator of the dilation catheter in an expanded state;

FIG. 12A depicts a cross-sectional rear view of the dilator of FIG. 11, taken along line 12-12 of FIG. 11;

FIG. 12B depicts an alternative cross-sectional rear view of the dilator of FIG. 11, taken along line 12-12 of FIG. 11;

FIG. 12C depicts another alternative cross-sectional rear view of the dilator of FIG. 11, taken along line 12-12 of FIG. 11;

FIG. 13A depicts a schematic view, along an axial plane, of anatomical structures associated with a nasal cavity of a patient, with a distal portion of a first guidewire of an ENT compression instrument positioned between an inferior turbinate and the nasal septum;

FIG. 13B depicts a schematic view, along an axial plane, of the anatomical structures associated with the nasal cavity of the patient of FIG. 13A, with a guide catheter of the ENT compression instrument advanced along the guidewire of FIG. 13A to position a first distal portion of the guide catheter between the inferior turbinate and the nasal septum such that a second distal portion of the guide catheter faces toward the inferior nasal meatus between the inferior turbinate and the lateral nasal wall;

FIG. 13C depicts a schematic view, along an axial plane, of the anatomical structures associated with the nasal cavity of the patient of FIG. 13A, with a second guidewire of the ENT compression instrument advanced through the guide catheter of FIG. 13B to position a distal portion of the second guidewire between the inferior turbinate and the lateral nasal wall;

FIG. 13D depicts a schematic view, along an axial plane, of the anatomical structures associated with the nasal cavity of the patient of FIG. 13A, with a dilation catheter of the ENT compression instrument advanced along the second guidewire of FIG. 13C to position a dilator of the dilation catheter between the inferior turbinate and the lateral nasal wall, and with the dilator in a non-expanded state;

FIG. 13E depicts a schematic view, along an axial plane, of the anatomical structures associated with the nasal cavity of the patient of FIG. 13A, with the dilator of FIG. 13D in an expanded state;

FIG. 14 depicts a schematic view, along an axial plane, of anatomical structures associated with a nasal cavity of a patient, with an articulatable paddle of an ENT compression instrument positioned between an inferior turbinate and the nasal septum and a dilator of the ENT compression instrument positioned between the inferior turbinate and the lateral nasal wall, and with the dilator in an expanded state;

FIG. 15 depicts a front elevation view of a distal portion of another exemplary dilation catheter having a dilator that includes a pair of laterally-opposed balloons, with the dilator in an expanded state;

FIG. 16A depicts a partial cross-sectional side view of a distal portion of another exemplary dilation catheter having a dilator at least partially constrained within a sheath, with the dilator in a non-expanded state;

FIG. 16B depicts a partial cross-sectional side view of the distal portion of the dilation catheter of FIG. 16A, with the dilator in an expanded state such that a protruding portion of the dilator bulges laterally outwardly through a lateral bore of the sheath;

FIG. 17 depicts a side elevational view of a distal portion of another exemplary dilation catheter having a dilator that includes a balloon and a helical wire;

FIG. 18 depicts a side elevational view of a distal portion of another exemplary dilation catheter having a dilator that includes a balloon and a pair of linear wires;

FIG. 19 depicts a side elevational view of a distal portion of another exemplary dilation catheter having a dilator that includes a spherical balloon and a hemispherical expandable basket;

FIG. 20 depicts a side elevational view of a distal portion of another exemplary dilation catheter having a dilator that includes a hemispherical balloon and a hemispherical expandable basket;

FIG. 21 depicts a side elevational view of a distal portion of another exemplary dilation catheter having a dilator that includes a spherical balloon and a spherical expandable basket having first and second hemispherical basket portions;

FIG. 22 depicts a side elevational view of a distal portion of another exemplary dilation catheter having a dilator that includes a spherical balloon having first and second hemispherical balloon portions;

FIG. 23A depicts a schematic view, along an axial plane, of anatomical structures associated with a nasal cavity of a patient, with the dilator of FIG. 22 positioned between an inferior turbinate and the lateral nasal wall, and with the dilator in a non-actuated, expanded state; and

FIG. 23B depicts a schematic view, along an axial plane, of the anatomical structures associated with the nasal cavity of the patient of FIG. 23A, with the dilator of FIG. 22 positioned between the inferior turbinate and the lateral nasal wall, and with the dilator in an actuated, expanded state.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. For example, while various. Accordingly, the drawings and descriptions should be regarded as illustrative in nature and not restrictive.

It will be appreciated that the terms “proximal” and “distal” are used herein with reference to a clinician gripping a handpiece assembly. Thus, an end effector is distal with respect to the more proximal handpiece assembly. It will be further appreciated that, for convenience and clarity, spatial terms such as “top” and “bottom” also are used herein with respect to the clinician gripping the handpiece assembly. However, surgical instruments are used in many orientations and positions, and these terms are not intended to be limiting and absolute.

It is further understood that any one or more of the teachings, expressions, versions, examples, etc. described herein may be combined with any one or more of the other teachings, expressions, versions, examples, etc. that are described herein. The following-described teachings, expressions, versions, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

### I. Exemplary Dilation Catheter

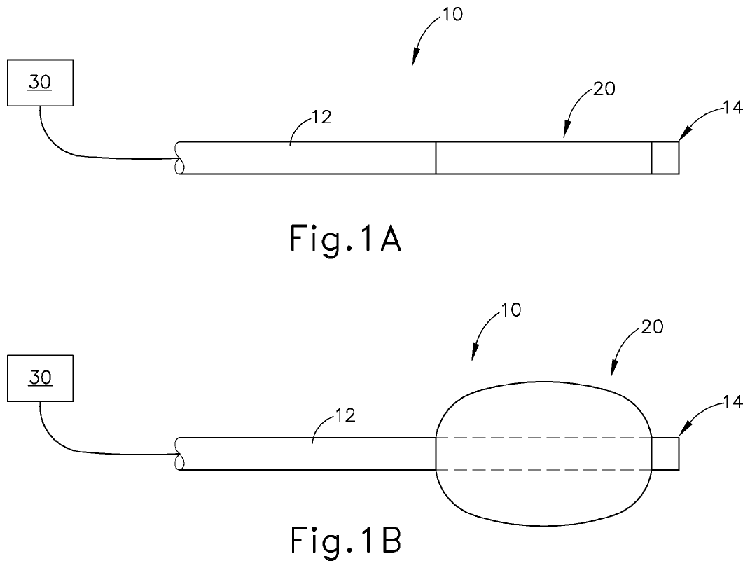

FIGS. 1A-1B show a distal portion of an exemplary dilation catheter (10). Dilation catheter (10) of this example includes an elongate shaft (12), with a dilator (20) positioned near the distal end (14) of shaft (12). Shaft (12) of the present example is generally flexible, such that distal end (14) and other portions of shaft (12) may bend away from a straight longitudinal axis of shaft (12). However, shaft (12) also has sufficient column strength to enable a distal portion of shaft (12) to be pushed into a nasal cavity of a patient (e.g., as described below), without causing shaft (12) to substantially buckle. Various suitable materials that may be used to form shaft (12) will be apparent to those of ordinary skill in the art in view of the teachings herein.

Dilator (20) of the present example comprises an inflatable balloon. Dilator (20) is in fluid communication with a source (30) of inflation fluid (e.g., saline). The inflation fluid may thus be communicated from source (30) to dilator (20) to transition dilator (20) from a non-expanded state (FIG. 1A) to an expanded state (FIG. 1B); and back from dilator (20) to source (30) to transition dilator (20) from the expanded state (FIG. 1B) back to the non-expanded state (FIG. 1A). In some versions, the balloon forming dilator (20) comprises an extensible material, such that dilator (20) is resiliently biased to assume the non-expanded configuration of FIG. 1A. In some other versions, the balloon forming dilator comprises a flexible yet non-extensible material (e.g., mylar). In some other versions, dilator (20) is in the form of a mechanically expandable element that does not require fluid to transition from a non-expanded state to an expanded state. In the present example, dilator (20) is configured to achieve an outer diameter of approximately 16 mm when dilator (20) is in the fully expanded state. By way of further example only, dilator (20) may be configured to achieve an outer diameter between approximately 10 mm and approximately 16 mm when dilator (20) is in the fully expanded state.

Shaft (12) of the present example further includes a lumen (not shown) providing a pathway for fluid communication between fluid source (30) and dilator (20). In some versions, shaft (12) also includes a separate lumen that is configured to slidably receive a guidewire. In addition, or in the alternative, shaft (12) may include one or more lumens that is/are configured to provide ventilation, suction, irrigation, medication, or other effects through distal end (14). Other features and operabilities that may be incorporated into dilation catheter (10) will be apparent to those of ordinary skill in the art in view of the teachings herein.

### II. Exemplary Method of Treating a Deviated Nasal Septum

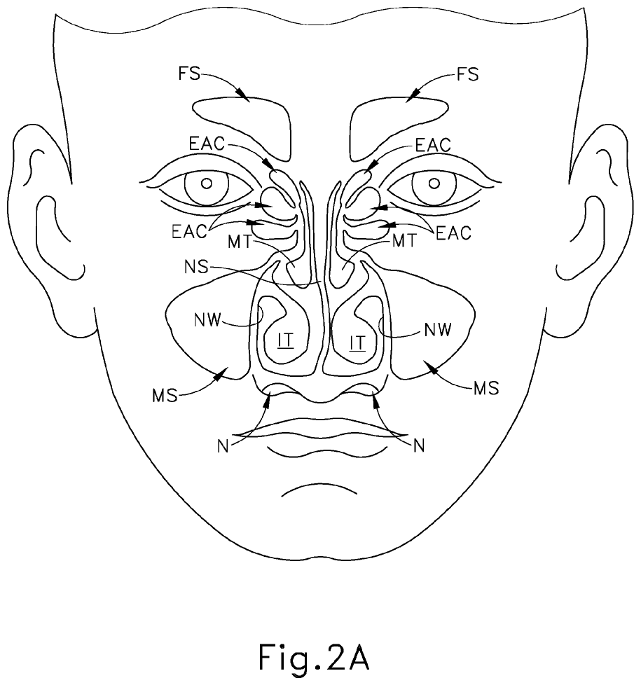

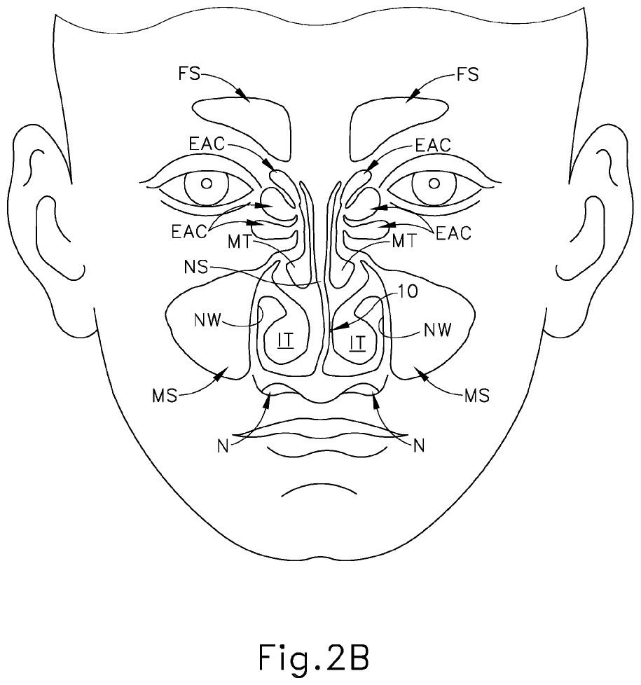

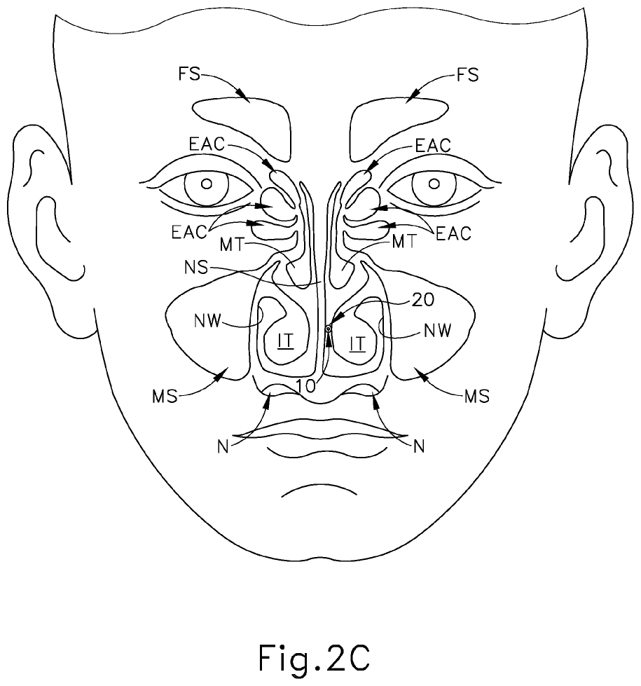

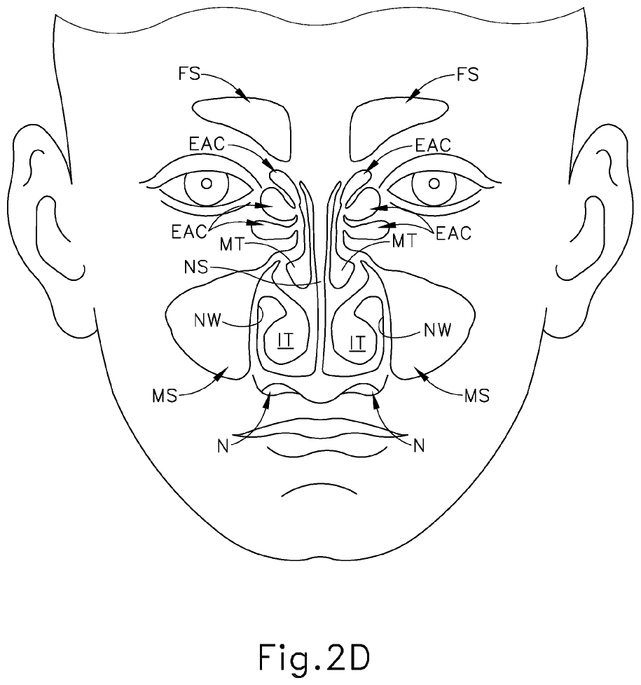

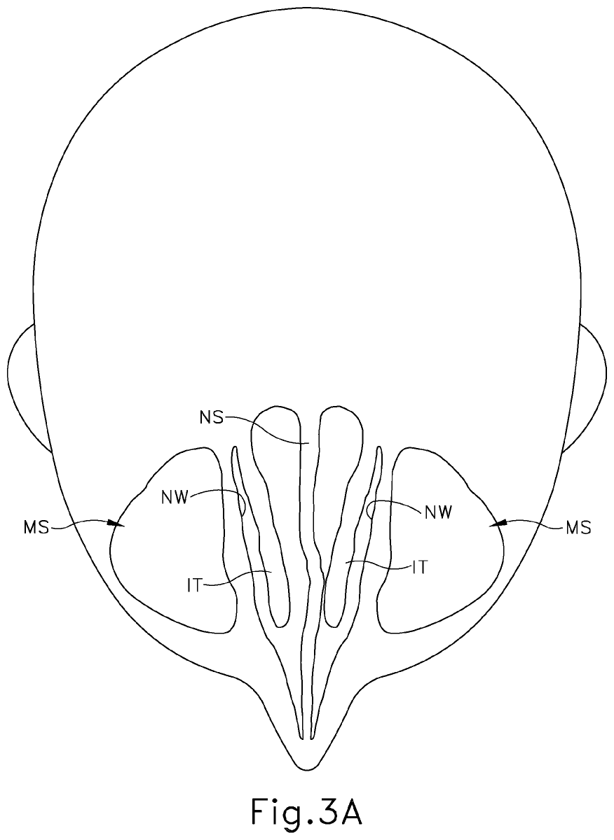

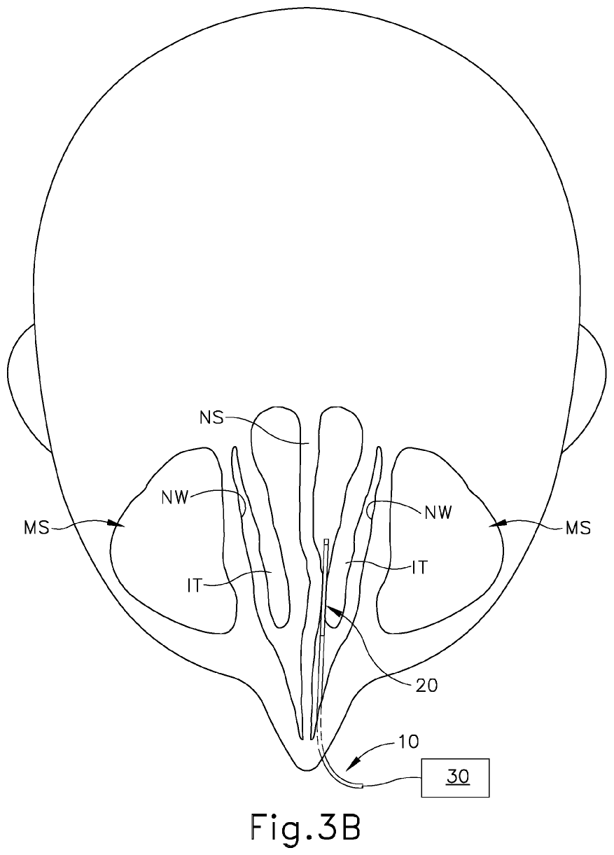

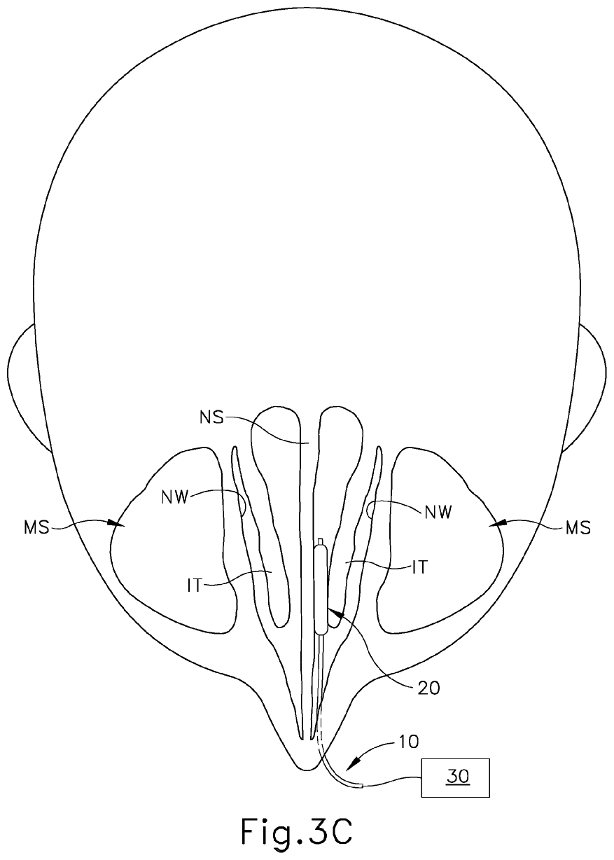

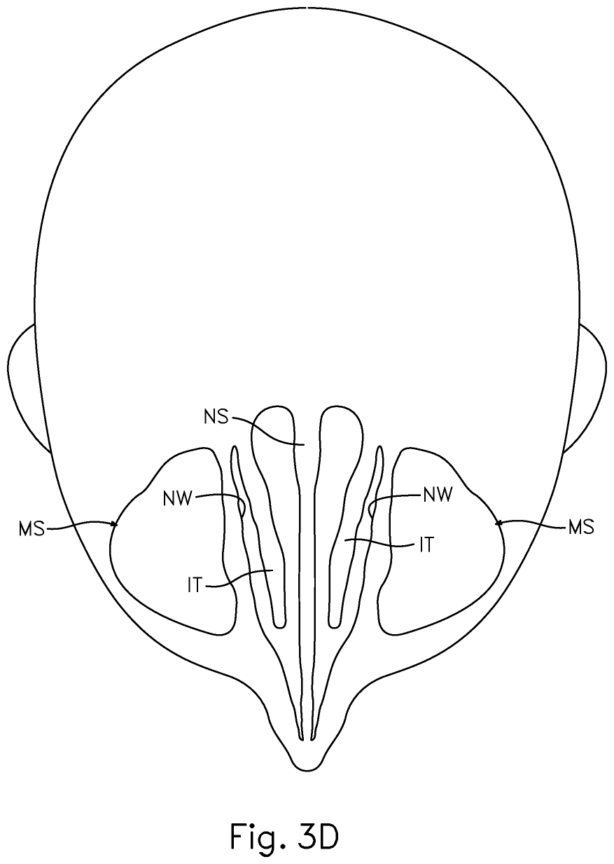

FIGS. 2A and 3A show various anatomical structures associated with a nasal cavity of a patient. These structures include a pair of frontal sinus cavities (FS), a set of ethmoid air cells (EAC), a pair of maxillary sinus cavities (MS), a pair of middle turbinates (MT), a pair of inferior turbinates (IT), and a nasal septum (NS) separating the members of each pair. Due to the location of the cross-sectional plane of the view in FIGS. 2A and 3A, the superior turbinates are not shown. As shown in FIGS. 2A and 3A, the nasal septum (NS) is deviated laterally against on inferior turbinate (IT) in the patient. As noted above, this condition may cause a restriction or blockage in the nasal passageway, which may lead to breathing difficulties, bleeding, pain, and/or other undesirable conditions in the patient.

FIGS. 2B and 3B show an initial step in an exemplary procedure to treat the deviated nasal septum (NS) of FIGS. 2A and 3A. In particular, the distal portion of dilation catheter (10) is inserted into a nostril (N) of the patient, on the side where the nasal septum (NS) is deviated into the inferior turbinate (IT). Dilator (20) is in a non-expanded state while dilation catheter (10) is inserted into position. Dilation catheter (10) is inserted to a position where dilator (20) is interposed between the deviated portion of the nasal septum (NS) and the inferior turbinate (IT). Shaft (12) of dilation catheter (10) provides sufficient column strength to overcome any frictional resistance provided between the nasal septum (NS) and the inferior turbinate (IT), thereby enabling dilator (20) to be positioned between the nasal septum (NS) and the inferior turbinate (IT) without causing substantial buckling in shaft (12).

In some variations, a guidewire (not shown) is first positioned between the nasal septum (NS) and the inferior turbinate (IT); and then dilation catheter (10) is advanced along the guidewire to position dilator (20) between the nasal septum (NS) and the inferior turbinate (IT). As another merely illustrative example, a rigid or malleable guide catheter may first be positioned at or in the nostril (N); and then dilation catheter (10) may be advanced through the guide catheter to position dilator (20) between the nasal septum (NS) and the inferior turbinate (IT). Other suitable devices and techniques that may be used to achieve the positioning shown in FIGS. 2B and 3B will be apparent to those of ordinary skill in the art in view of the teachings herein.

Once dilator (20) has been suitably positioned between the nasal septum (NS) and the inferior turbinate (IT), inflation fluid is driven from fluid source (30) to dilator (20), thereby expanding dilator (20) to the expanded state shown in FIGS. 2C and 3C. As dilator (20) expands, dilator (20) urges the nasal septum (NS) medially, thereby substantially straightening the nasal septum (NS). As the nasal septum (NS) is urged medially, the bone in the nasal septum (NS) may fracture and/or the cartilage in the nasal septum (NS) may plastically deform, such that the medially urged nasal septum (NS) is effectively remodeled and maintains a substantially straight configuration after dilation catheter (10) is removed as shown in FIGS. 2D and 3D.

In the present example, during the stage shown in FIGS. 2C and 3C, the adjacent inferior turbinate (IT) provides at least some degree of a mechanical ground for dilator (20), enabling the expanded dilator (20) to move the nasal septum (NS) medially. In some scenarios, the adjacent inferior turbinate (IT) is urged laterally (and, in some cases, at least partially fractured) to at least some degree when dilator (20) is expanded. In such scenarios, the inferior turbinate (IT) may still eventually engage the adjacent lateral nasal wall (NW), such that the laterally urged inferior turbinate (IT) cooperates with the adjacent lateral nasal wall (NW) to provide a mechanical ground for the expanded dilator (20). In addition to remodeling the nasal septum (NS) as described above, the expansion of dilator (20) may further remodel the adjacent inferior turbinate (IT) to some degree. For instance, the expanding dilator (20) may fracture at least some of the bone forming the inferior turbinate (IT), such that the inferior turbinate (IT) remains at least partially lateralized after dilation catheter (10) is removed from the nasal cavity. Thus, while FIGS. 2D and 3D only shows the nasal septum (NS) being remodeled at the end of the procedure of FIGS. 2A-2D and 3A-3D, the inferior turbinate (IT) may also be remodeled at the end of the procedure in some scenarios. Moreover, the mucosa of the inferior turbinate (IT) may be crushed during the procedure.

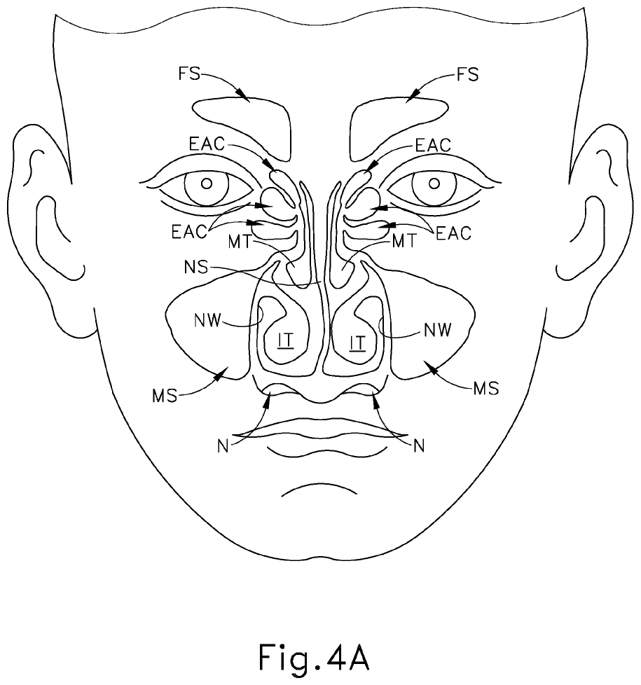

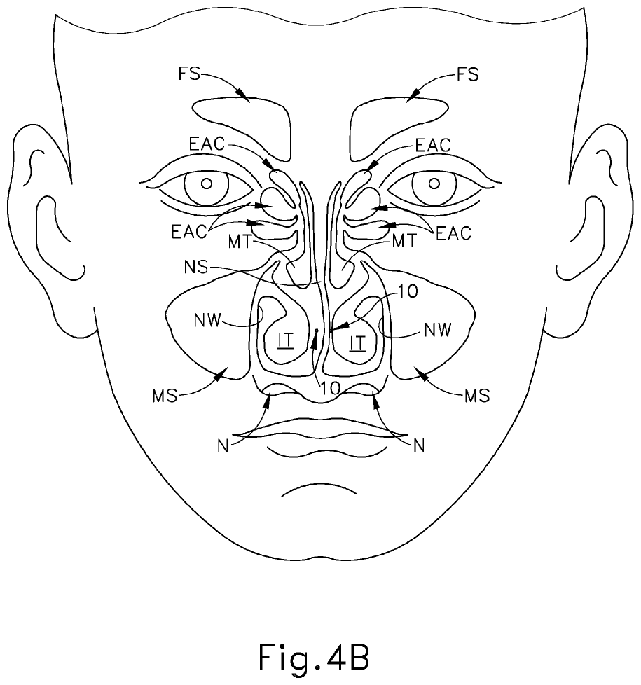

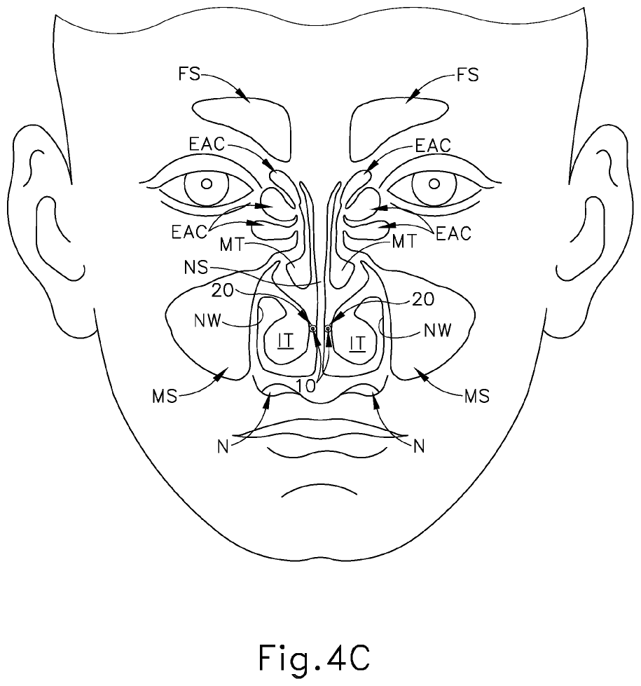

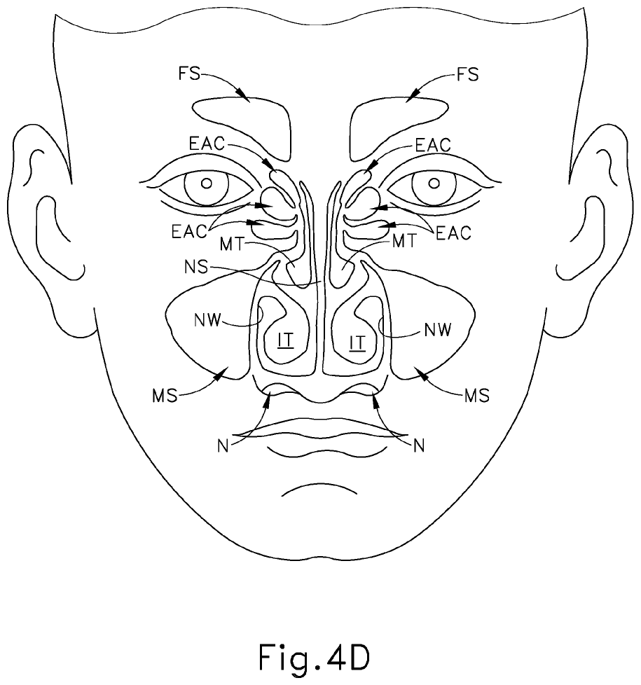

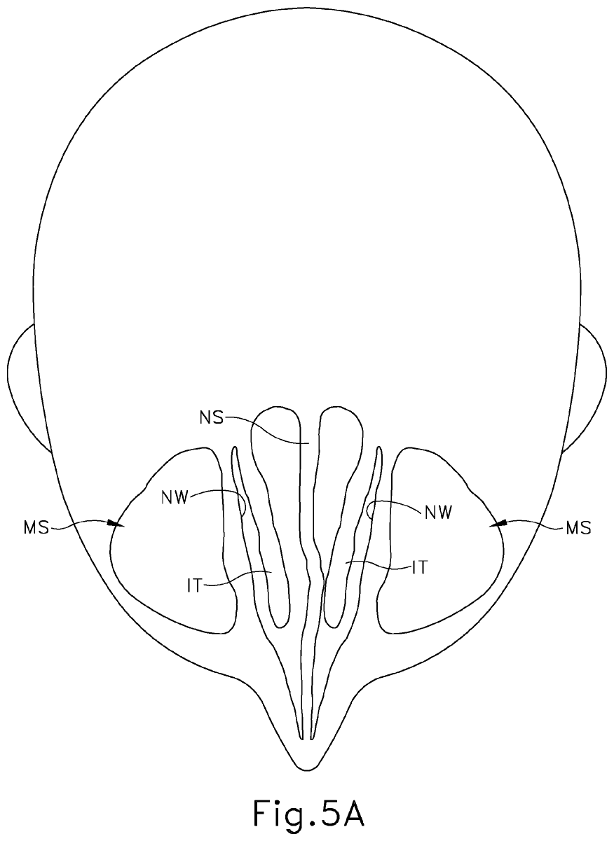

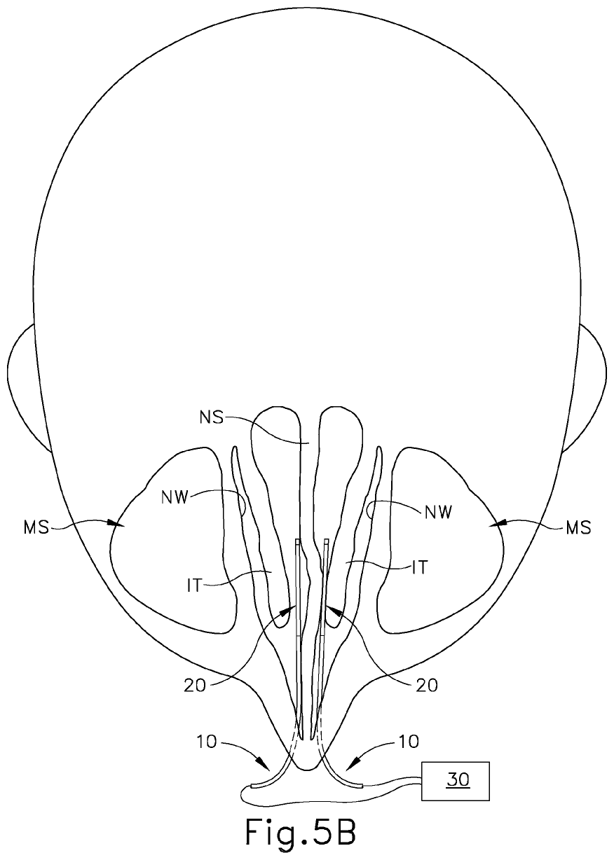

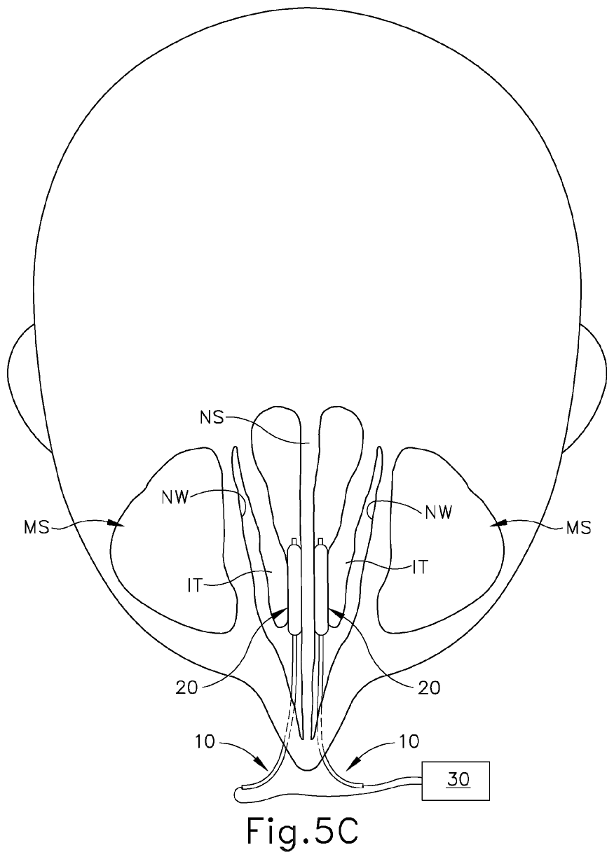

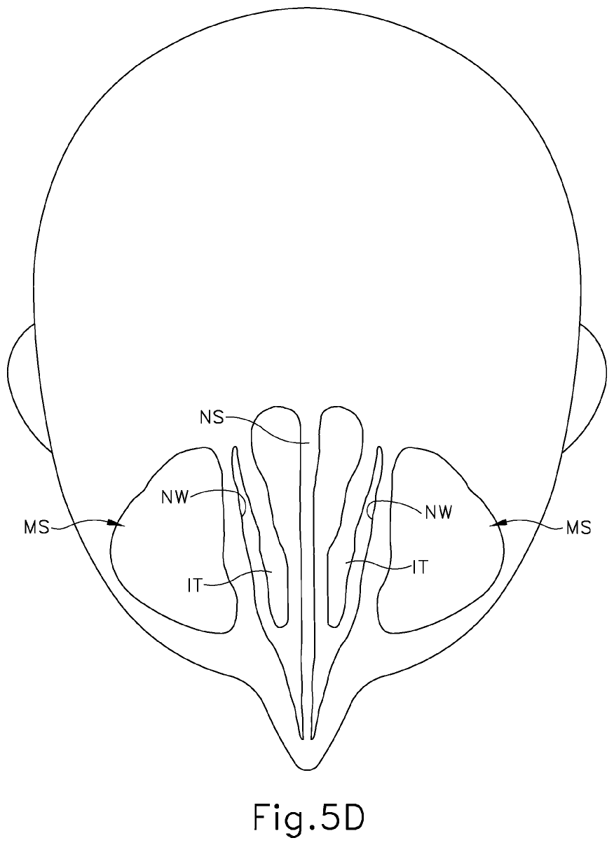

FIGS. 4A-5D show an exemplary alternative procedure that may be used to treat a deviated nasal septum (NS). As shown in FIGS. 4A and 5A, the patient has the same deviated nasal septum (NS) state as the patient shown in FIGS. 2A and 3A. In this alternative treatment procedure, two dilation catheters (10) are used. As shown in FIGS. 4B and 5B, a dilation catheter (10) is inserted into each nostril (N), with both dilators (20) in the non-expanded state. The distal portion of a first dilation catheter (10) is inserted into the nostril (N) of the patient on the side where the nasal septum (NS) is deviated into the inferior turbinate (IT). This first dilation catheter (10) is inserted to a position where dilator (20) is interposed between the deviated portion of the nasal septum (NS) and the inferior turbinate (IT). The distal portion of the second dilation catheter (10) is inserted into the other nostril (N), to a depth corresponding to the insertion depth of the first dilation catheter (10). Both dilation catheters (10) are thus correspondingly positioned on opposite sides of the nasal septum (NS). As noted above, guidewires, guide catheters, and/or any other suitable devices or techniques may be used to assist in achieving the positioning shown in FIGS. 4B and 5B.

Once dilators (20) have been suitably positioned as shown in FIGS. 4B and 5B, inflation fluid is driven from fluid source (30) to dilators (20), thereby expanding dilators (20) to the expanded state shown in FIGS. 4C and 5C. In the present example, both dilators (20) are expanded simultaneously. In some other versions, the dilator (20) on the left in the view shown in FIGS. 4B and 5B (i.e., the patient's right side) is expanded first; followed by the dilator (20) on the right in the view shown in FIGS. 4B and 5B (i.e., the patient's left side). Also in the present example, both dilators (20) are coupled with the same fluid source (30). In some other versions, each dilator (20) has its own respective fluid source (30). In either case, as the dilator (20) on the right in the view shown in FIGS. 4B and 5B (i.e., the patient's left side) expands, dilator urges the nasal septum (NS) medially, thereby substantially straightening the nasal septum (NS). As the nasal septum (NS) is urged medially, the bone in the nasal septum (NS) may fracture and/or the cartilage in the nasal septum (NS) may plastically deform, such that the medially urged nasal septum (NS) is effectively remodeled and maintains a substantially straight configuration after dilation catheter (10) is removed as shown in FIGS. 4D and 5D.

In the present example, the expanded dilator (20) on the left side in the view shown in FIGS. 4C and 5C (i.e., the patient's right side) may provide a stop for the medialized nasal septum (NS), thereby preventing over-medialization of the nasal septum (NS). In other words, in some scenarios where a procedure is performed as shown in FIGS. 2A-3D with just one dilator (20), dilator (20) may urge the nasal septum (NS) too far medially, to the point where the nasal septum (NS) is transitioned from deviating too far to the patient's left side to deviating too far to the patient's right side, when the goal of the procedure is to achieve a substantially straight nasal septum (NS). Thus, by providing an opposing expanded dilator (20) as shown in FIGS. 4C and 5C, the expanded dilator (20) on the patient's right side may prevent the nasal septum (NS) from being deformed right-of-center by the expanded dilator (20) on the patient's left side. In other words, using two opposing dilators (20) on opposing sides of the nasal septum (NS) may ensure that the nasal septum (NS) is not deformed beyond the substantially straight position shown in FIGS. 4D and 5D. Using two opposing dilators (20) on opposing sides of the nasal septum (NS) may also increase the effect that dilator (20) has on reducing mucosal hypertrophy.

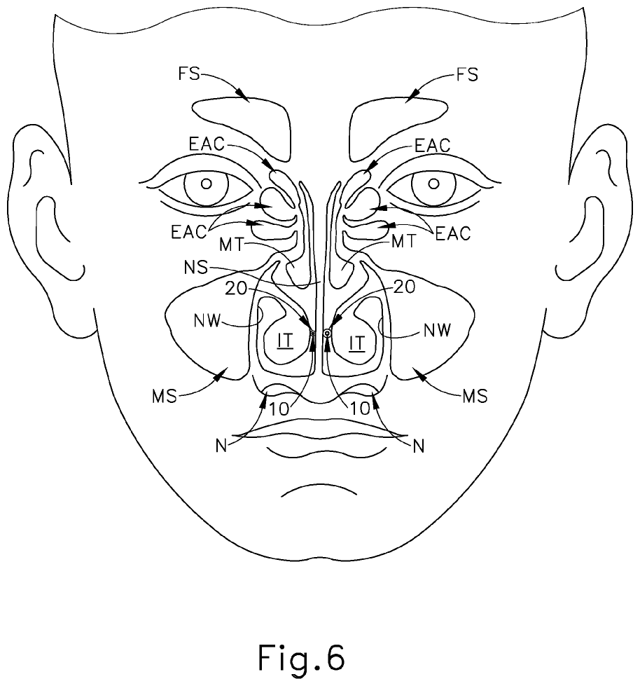

In the example shown in FIGS. 4C and 5C, both dilators (20) are expanded to approximately the same outer diameter. In some other versions, dilators (20) are expanded to different outer diameters, as shown in FIG. 6. By way of example only, in some variations the dilator (20) on the side to which the nasal septum (NS) is deviated (i.e., the patient's left side in the views shown in FIGS. 4A-5D) may be expanded to a larger outer diameter; while the dilator (20) on the opposite side (i.e., the patient's left side in the views shown in FIGS. 4A-5D) may be expanded to a smaller outer diameter). By way of further example only, the larger outer diameter may be approximately 16 mm while the smaller outer diameter may be approximately 10 mm.

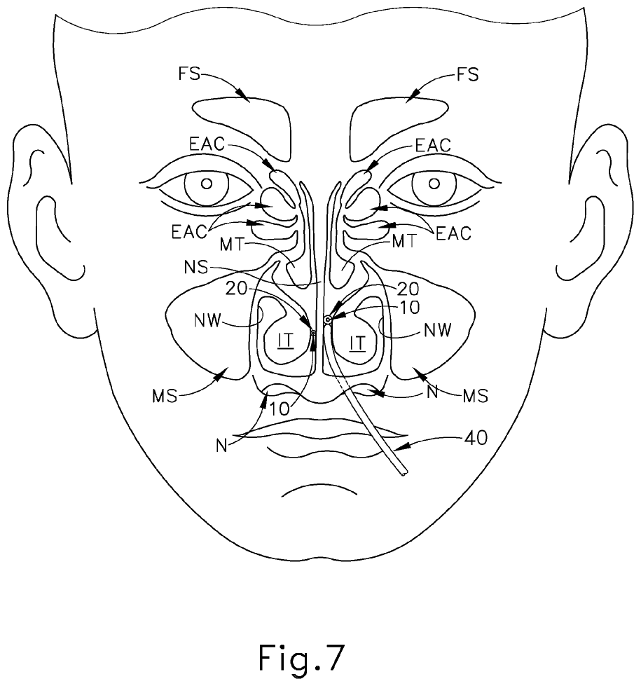

As another merely illustrative variation, dilators (20) may be positioned at different vertical heights within the nasal cavity. For instance, a spacer device (40) may be inserted into a nostril and be used to urge a dilator (20) superiorly, with the dilator (20) on the other side of the nasal septum (NS) being positioned inferiorly relative to the superiorly raised dilator (20). A merely illustrative example of such positioning is shown in FIG. 7. Various suitable forms that spacer device (40) may take will be apparent to those of ordinary skill in the art in view of the teachings herein.

In many of the foregoing examples, the nasal septum (NS) is laterally deviated near the inferior turbinate (IT), such that dilation catheter (10) is positioned to locate dilator (20) between the nasal septum (NS) and the inferior turbinate (IT). In some other scenarios, the nasal septum (NS) laterally deviated near the middle turbinate (MT). In such scenarios, the dilation catheter (10) may be positioned to locate dilator (20) between the nasal septum (NS) and the middle turbinate (MT). Likewise, the dilation catheter (10) may be positioned to locate dilator (20) between the nasal septum (NS) and the superior turbinate (not shown) in scenarios where the nasal septum (NS) is laterally deviated near the superior turbinate.

While the foregoing examples are provided in the context of treating a deviated nasal septum (NS), the procedures identified above may be modified to treat other conditions within the nasal cavity. For instance, dilation catheter (10) may be used to remodel an enlarged turbinate (MT, IT), by placing dilator (20) against the enlarged turbinate (MT, IT) and then expanding dilator (20) to remodel the enlarged turbinate (MT, IT). In such procedures, depending on which side of the turbinate (MT, IT) the dilator (20) is positioned, the lateral nasal wall (NW) or the nasal septum (NS) may provide a mechanical ground for the expanding dilator (20). In such procedures where the nasal septum (NS) is used to provide a mechanical ground, including cases where the nasal septum (NS) is not deviated at all, it may be advantageous to provide an opposing dilator (20) on the opposite side of the nasal septum (NS). This may help shore up the nasal septum (NS) and thereby prevent undesired remodeling of the nasal septum (NS) when the nasal septum (NS) is used to provide a mechanical ground in a procedure for remodeling a turbinate (MT, IT) with a dilator (20).

In addition to, or as an alternative to, remodeling the nasal septum (NS) and/or a turbinate (MT, IT), an expanded dilator (20) may move and/or remodel mucosal tissue in the nasal cavity, which may further promote better airflow through the nasal cavity. For instance, as noted above, an expanded dilator (20) may crush the mucosal tissue that lines a passageway within the nasal cavity, thereby providing a wider pathway for airflow through that passageway.

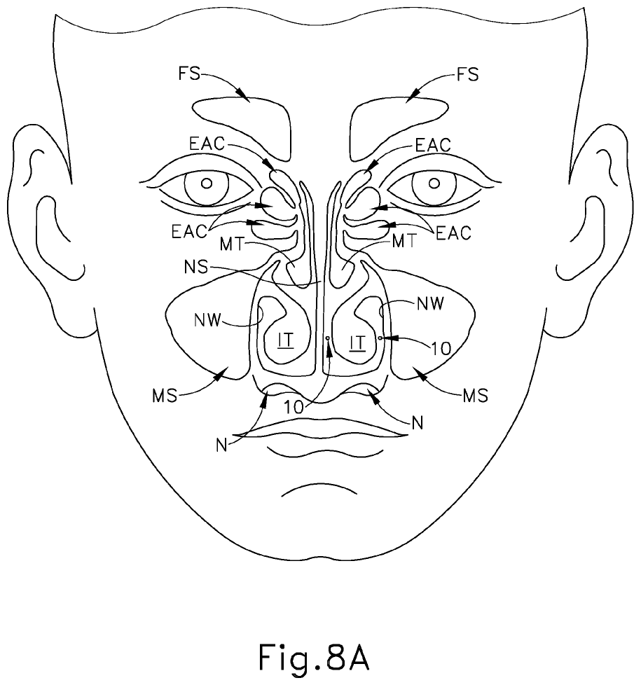

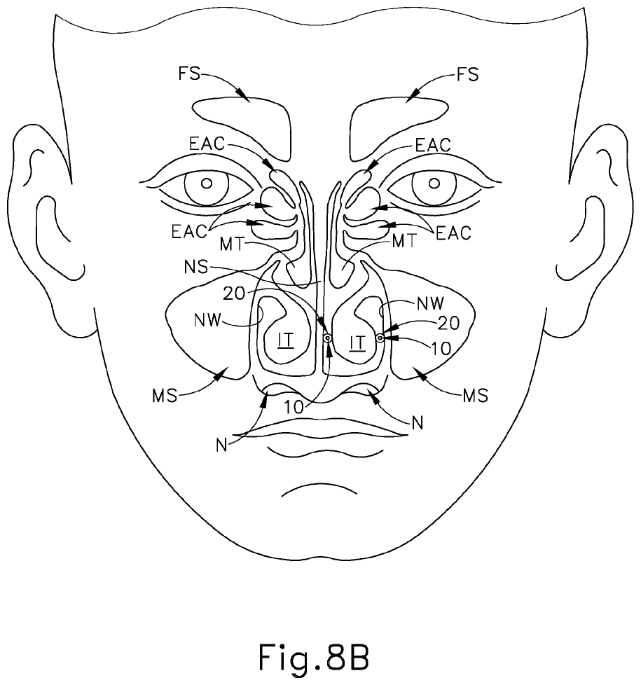

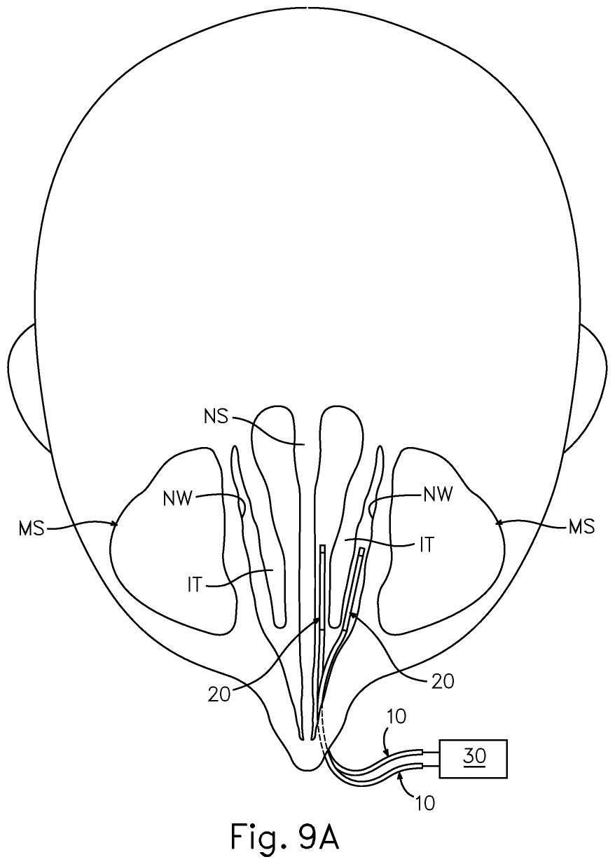

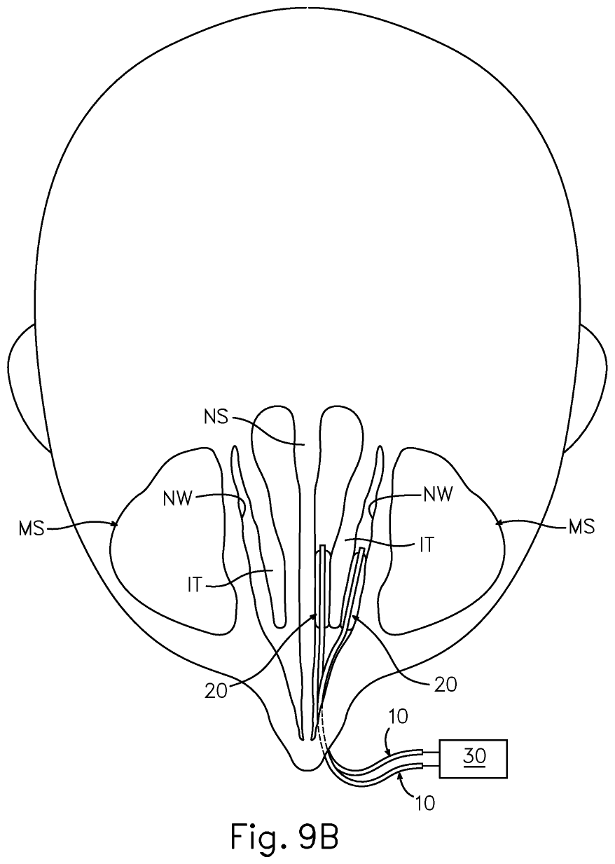

As yet another merely illustrative example, a first dilator (20) may be positioned between the nasal septum (NS) and the inferior turbinate (IT), with a second dilator (20) being positioned between the inferior turbinate (IT) and the lateral nasal wall (NW) (e.g., in the inferior nasal meatus) as shown in FIGS. 8A and 9A. When these dilators (20) are expanded simultaneously as shown in FIGS. 8B and 9B, this may prevent or otherwise reduce fracturing of bone within the inferior turbinate (IT), while still providing crushing of the mucosa of the inferior turbinate (IT). In some implementations of this procedure, the dilator (20) that is positioned between the inferior turbinate (IT) and the lateral nasal wall (NW) is expanded to an outer diameter that is smaller than the outer diameter to which the dilator (20) between the nasal septum (NS) and the inferior turbinate (IT) is expanded.

In the present example, both dilators (20) are expanded to substantially the same outer diameter at the stage shown in FIGS. 8B and 9B. Alternatively, dilators (20) may be expanded to different outer diameters at the stage shown in FIGS. 8B and 9B. By way of example only, the dilator (20) that is positioned between the inferior turbinate (IT) and the lateral nasal wall (NW) may be expanded to an outer diameter of approximately 10 mm; and the dilator (20) that is positioned between the nasal septum (NS) and the inferior turbinate (IT) may be expanded to an outer diameter of approximately 16 mm. This procedure (and any other procedure directed to treatment of the inferior turbinate (IT) and/or mucosal tissue) may be performed in cases where the patient does not have a deviated nasal septum (NS). For example, the procedure depicted in FIGS. 8A-9B may result in crushing of one or more posterior nasal nerves sufficient to effectively disable the posterior nasal nerve, such that dilators (20) provide denervation in addition to, or in lieu of, remodeling the inferior turbinate (IT). Thus, the procedures described herein are not limited to scenarios where the patient has a deviated nasal septum (NS).

### III. Exemplary Alternative Dilation Catheters

In some instances, when dilator (20) is inflated in accordance with the description above, dilator (20) may “slip” or otherwise move relative to adjacent anatomical structures in response to contact between dilator (20) and adjacent anatomical structures. If dilator (20) “slips” in response to contact with adjacent anatomical structures during inflation, dilator (20) may not be located in the desired location when fully inflated. If dilator (20) slips during inflation, dilator (20) may thus fail to suitably dilate the targeted anatomical structure. It may therefore be desirable to modify dilator (20) to prevent dilator (20) from slipping relative to adjacent anatomical structure during inflation. Adding a textured outer surface, a non-circular cross-sectional profile, and/or some other kind of friction enhancing feature to dilator (20) may help prevent dilator (20) from slipping relative to adjacent anatomical structures as dilator (20) comes into contact with adjacent anatomical structures during inflation.

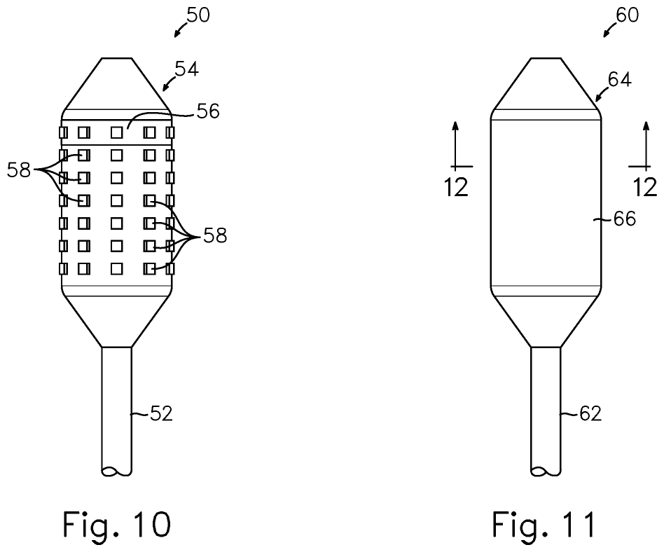

FIGS. 10 and 11 show alternative dilation catheters (50, 60) that may be readily used in replacement of dilation catheter (10) described above. Dilation catheters (50, 60) are substantially similar to dilation catheters (10) described above, with differences elaborated below. Dilation catheters (50, 60) include respective elongate shafts (52, 62) and dilators (54, 64), which may be substantially similar to elongate shafts (12) and dilator (20) described above, with differences described below. As will be described in greater detail below, each dilator (54, 64) includes a friction enhanced feature configured to prevent slipping of dilators (54, 64) during inflation.

Dilator (54) includes a primary exterior surface (56) and a plurality of secondary protrusions (58) extending from primary exterior surface (56). Primary exterior surface (56) may form a portion of dilator (54) that is substantially similar to dilator (20) described above, while secondary protrusions (58) may create a textured or patterned surface that may help prevent dilator (54) from slipping during inflation. Secondary protrusions (58) may be formed of a material that is different from primary exterior surface (56). Specifically, secondary protrusions (58) may be formed of a “rougher” material that has a greater coefficient of friction compared to primary exterior surface (56). The increased coefficient of friction of secondary protrusions (58) may help prevent dilator (54) from slipping relative to adjacent anatomical structures during inflation in exemplary use. Alternatively, secondary protrusions (58) may be made from the same material of primary exterior surface (56), and the geometry of secondary protrusions (58) may help increase to frictional gripping of dilator (54) with adjacent anatomical structures.

Dilator (64) includes a textured external surface (66) formed of a “rougher” material, such as a rough silicone surface. The roughened surface of textured external surface (66) may provide an increased coefficient of friction compared to dilator (20) described above. This increase in the coefficient of friction may help prevent dilator (64) from slipping relative to adjacent anatomical structures during inflation in exemplary use.

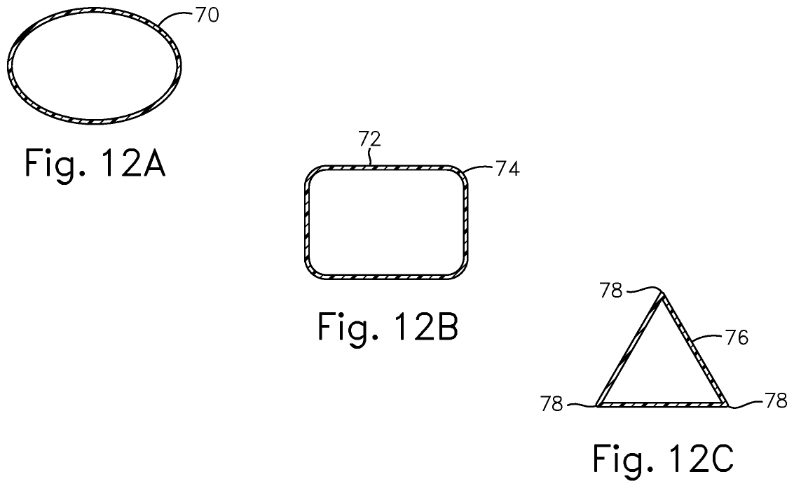

FIGS. 12A-12C show various cross-sectional profiles that may be readily incorporated into dilators (20, 54, 64) in order to help prevent slippage of dilators (20, 54, 64) during inflation. FIG. 12A shows a substantially oval profile (70). FIG. 12B shows a rectangular profile (72) having rounded corners (74). FIG. 12C shows a triangular profile (76) having rounded corners (78). The change in cross-sectional profile, compared to a circular profile, may help prevent dilators (20, 54, 64) from slipping during inflation by limiting points of contact between dilators (20, 54, 64) and adjacent anatomical structures.

In addition to the foregoing, dilators (20, 54, 64) may be constructed and operable in accordance with at least some of the teachings of U.S. Pat. Pub. No. 2014/0277071, entitled “Features to Enhance Grip of Balloon Within Airway,” published Sep. 18, 2014, now abandoned, the disclosure of which is incorporated by reference herein, in its entirety.

### IV. Exemplary ENT Instruments for Compressing a Nasal Nerve

It will be appreciated that the vidian nerve resides within the vidian (or “pterygoid”) canal, which is defined in part by the sphenoid bone and is located posterior to the sphenoid sinus, approximately in alignment with the middle turbinate (MT). The vidian nerve is formed at its posterior end by the junction of the greater petrosal nerve and the deep petrosal nerve; and joins at its anterior end with the pterygopalatine ganglion, which is responsible for regulating blood flow to the nasal mucosa. The posterior nasal nerves join with the pterygopalatine ganglion and extend through the region surrounding the inferior turbinate (IT). In some instances, it may be desirable to compress a nasal nerve, such as a posterior nasal nerve as an alternative to a traditional vidian neurectomy procedure. For example, referring again to FIGS. 8A and 9A, the expansion of first and second dilators (20) positioned between the nasal septum (NS) and the inferior turbinate (IT) and between the inferior turbinate (IT) and the lateral nasal wall (NW), respectively, may in some cases be sufficient to compress the posterior nasal nerve within, extending through, surrounding, or otherwise associated with the inferior turbinate (IT).

Nevertheless, it may be desirable to provide an ENT compression instrument having a dilator that is selectively actuatable between non-expanded and expanded states to facilitate effective and safe compression of a nasal nerve, such as a posterior nasal nerve, while minimizing or preventing undesired remodeling of the nasal septum (NS) and/or lateral nasal wall (NW). Each of the exemplary ENT compression instruments (110, 210) and dilation catheters (112, 212, 312, 412, 512, 612, 712, 812, 912, 1012) described below may function in such a manner. While the examples provided below are discussed in the context of posterior nasal nerve compression, ENT compression instruments (110, 210) and dilation catheters (112, 212, 312, 412, 512, 612, 712, 812, 912, 1012) may be used to compress tissue in various other regions within the ear, nose, or throat of a patient. ENT compression instruments (110, 210) and dilation catheters (112, 212, 312, 412, 512, 612, 712, 812, 912, 1012) may also be used to remodel anatomical structures in addition to, or in lieu of, affecting a posterior nasal nerve or other nerve structure. Other suitable ways in which ENT compression instruments (110, 210) and dilation catheters (112, 212, 312, 412, 512, 612, 712, 812, 912, 1012) may be used will be apparent to those skilled in the art in view of the teachings herein.

A. Exemplary ENT Compression Instrument with Bifurcated Guide Catheter

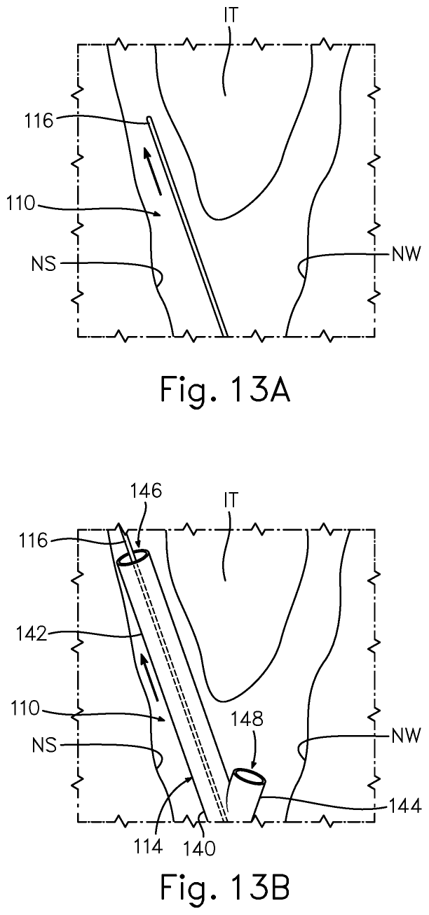

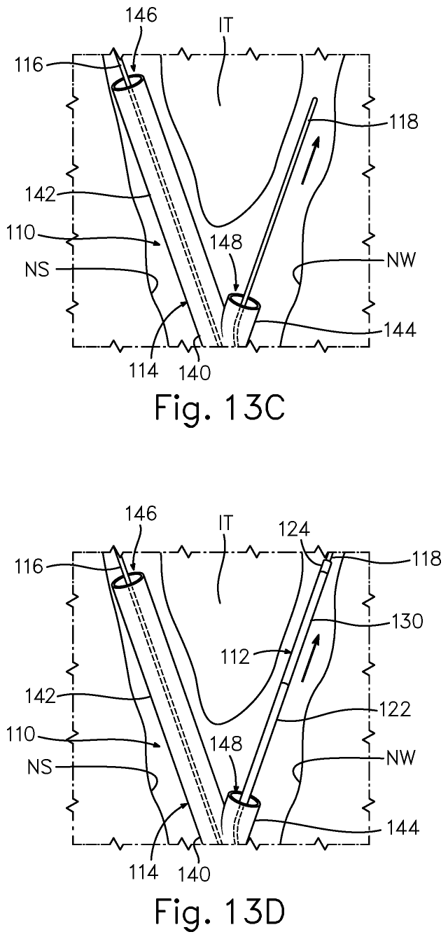

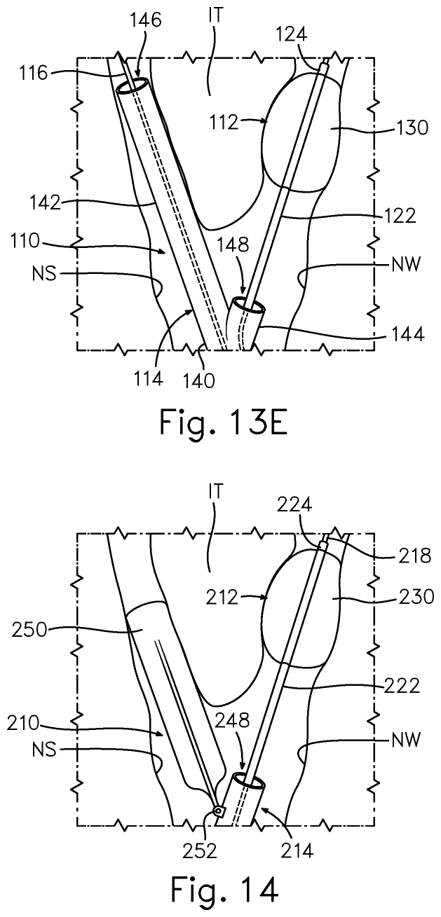

FIGS. 13A-13E show a distal portion of an exemplary ENT compression instrument (110) that may be used to compress a posterior nasal nerve or some other anatomical structure (e.g., within the ear, nose, or throat, etc.). Compression instrument (110) of this example comprises a dilation catheter (112), a rigid, malleable, or steerable guide catheter (114), a first guidewire (116) slidably disposed within guide catheter (114), and a second guidewire (118) slidably disposed within dilation catheter (112).

Dilation catheter (112) of this example includes an elongate shaft (122) having a distal end (124), with a dilator (130) positioned at or near distal end (124) of shaft (122). Shaft (122) of the present example is generally flexible, such that distal end (124) and other portions of shaft (122) may bend away from a straight longitudinal axis of shaft (122). However, shaft (122) also has sufficient column strength to enable a distal portion of shaft (122) to be pushed into a nasal cavity of a patient (e.g., as described below), without causing shaft (122) to substantially buckle. Various suitable materials that may be used to form shaft (122) will be apparent to those of ordinary skill in the art in view of the teachings herein.

Dilator (130) of the present example comprises an inflatable balloon. An interior of dilator (130) is in fluid communication with a source (not shown) of inflation fluid (e.g., saline). The inflation fluid may thus be communicated from the inflation fluid source to dilator (130) to transition dilator (130) from a non-expanded state (FIG. 13D) to an expanded state (FIG. 13E); and back from dilator (130) to the inflation fluid source to transition dilator (130) from the expanded state (FIG. 13E) back to the non-expanded state (FIG. 13D). In some versions, the balloon forming dilator (130) comprises an extensible material, such that dilator (130) is resiliently biased to assume the non-expanded state of FIG. 13D. In some other versions, the balloon forming dilator (130) comprises a flexible yet non-extensible material (e.g., mylar). In some other versions, dilator (130) is in the form of a mechanically expandable element that does not require fluid to transition from a non-expanded state to an expanded state. In still other versions, dilator (130) is configured and operable like any of the other expandable dilation structures described below.

Shaft (122) of the present example further includes a first lumen (not shown) providing a pathway for fluid communication between the inflation fluid source and dilator (130). Shaft (122) also includes a second lumen (not shown) that is configured to slidably receive second guidewire (118). In some versions, second guide wire (118) is omitted. In some such versions, the second lumen may be used to provide irrigation, suction, a passageway for another instrument, or for any other suitable purpose(s). In still other versions, the second lumen is omitted from shaft (122).

Guide catheter (114) of the present example includes a proximal portion (140), a first distal portion (142) extending distally from a distal end of proximal portion (140), and a second distal portion (144) extending at least slightly outwardly (e.g., distally and/or laterally) from the distal end of proximal portion (140). In this manner, guide catheter (114) may be generally Y-shaped and may be considered bifurcated. In some other versions, guide catheter (114) includes a transversely oriented opening instead of including second distal portion (144), such that guide catheter (114) does not necessarily need to define a Y-shaped configuration. Guide catheter (114) of the present example defines a first lumen (not shown) extending along proximal portion (140) and first distal portion (142) to a first open distal end (146). The first lumen of guide catheter (114) is configured to slidably receive first guidewire (116). Guide catheter (114) also defines a second lumen (not shown) extending along proximal portion (140) and second distal portion (144) to a second open distal end (148). The second lumen of guide catheter (114) is configured to slidably receive second guidewire (118) and dilation catheter (112), such that guide catheter (114) may guide each of second guidewire (118) and dilator (130) out through second open distal end (148). In some versions, the first and second lumens may be isolated from each other.

In the example shown, first distal portion (142) is substantially coaxial with proximal portion (140), while second distal portion (144) extends obliquely relative to a longitudinal axis of proximal portion (140). As shown, first distal portion (142) of guide catheter (114) also extends distally beyond second distal portion (144) of guide catheter (114), such that second open distal end (148) of guide catheter (114) is positioned proximally relative to first open distal end (146). In this manner, second open distal end (148) may be configured to face toward the inferior nasal meatus between the inferior turbinate (IT) and the lateral nasal wall (NW) while first distal portion (142) is positioned between the nasal septum (NS) and the inferior turbinate (IT), as described in greater detail below. In some versions, guide catheter (114) may have a sufficient degree of stiffness to resist deflection of guide catheter (114) during expansion of dilator (130) to the expanded state for applying pressure to the posterior nasal nerve surrounding the inferior turbinate (IT).

Each guidewire (116, 118) of the present example may comprise a coil (not shown) positioned about a core wire (not shown). In some versions, one or both guidewires (116, 118) may have a sufficient degree of stiffness to resist deflection of the respective guidewire (116, 118) during expansion of dilator (130) to the expanded state for applying pressure to the posterior nasal nerve surrounding the inferior turbinate (IT). One or both of guidewires (116, 118) may also have one or more position sensors that is/are operable to generate signals indicating a real-time position of guidewire (116, 118) in three-dimensional space. By way of example only, one or both guidewires (116, 118) may be configured in accordance with at least some of the teachings of U.S. Pat. No. 10,463,242, entitled “Guidewire Navigation for Sinuplasty,” issued Nov. 5, 2019, the disclosure of which is incorporated by reference herein, in its entirety.

In an exemplary nasal nerve compression procedure, first guidewire (116) may initially be inserted into a nostril (not shown) of a patient and advanced distally through the nasal cavity to position a distal portion of first guidewire (116) between the nasal septum (NS) and the inferior turbinate (IT), as shown in FIG. 13A. Subsequently, the first lumen of guide catheter (114) may be advanced distally over first guidewire (116) to position first distal portion (142) of guide catheter (114) between the nasal septum (NS) and the inferior turbinate (IT), with second open distal end (148) of guide catheter (114) facing toward the inferior nasal meatus between the inferior turbinate (IT) and the lateral nasal wall (NW), as shown in FIG. 13B. In some variations of this process, first guidewire (116) is omitted, and guide catheter (114) is advanced to the position shown in FIG. 13B without using a guidewire. In some such variations, guide catheter (114) includes one or more position sensors that is/are operable to generate signals indicating a real-time position of guide catheter (114) in three-dimensional space. For instance, at least one position sensor may indicate the real-time position of second open distal end (148) in three-dimensional space. In addition, or in the alternative, at least one position sensor may indicate the real-time position of first open distal end (146) in three-dimensional space. In some other variations where first guidewire (116) is omitted, position sensors are also omitted.

Next, second guidewire (118) may be advanced distally through the second lumen of guide catheter (114) and out of second open distal end (148) of guide catheter (114) to position a distal portion of second guidewire (118) in the inferior nasal meatus between the inferior turbinate (IT) and the lateral nasal wall (NW), as shown in FIG. 13C. Dilation catheter (112) may then be advanced distally through the second lumen of guide catheter (114) along second guidewire (118) and out of second open distal end (148) of guide catheter (114) to position dilator (130) of dilation catheter (112) within the inferior nasal meatus between the inferior turbinate (IT) and the lateral nasal wall (NW) while dilator (130) is in the non-expanded state, as shown in FIG. 13D. In some versions, dilator (130) is inserted to a depth corresponding to the insertion depth of first distal portion (142) of guide catheter (114). Dilator (130) and first distal portion (142) of guide catheter (114) are thus correspondingly positioned on opposite sides of the inferior turbinate (IT). In some variations of this process, second guidewire (118) is omitted, and dilator (130) is advanced to the position shown in FIG. 13D without using a guidewire. In some such variations, dilation catheter (112) includes one or more position sensors that is/are operable to generate signals indicating a real-time position of dilation catheter (112) in three-dimensional space. For instance, at least one position sensor may indicate the real-time position of distal end (124) in three-dimensional space. In addition, or in the alternative, at least one position sensor may indicate the real-time position of dilator (130) in three-dimensional space.

After dilator (130) has been positioned between the inferior turbinate (IT) and the lateral nasal wall (NW), dilator (130) may be expanded to the expanded state for applying pressure to the posterior nasal nerve within, extending through, surrounding, or otherwise associated with the inferior turbinate (IT), as shown in FIG. 13E. More particularly, the expansion of dilator (130) urges the inferior turbinate (IT) medially, thereby compressing the inferior turbinate (IT) and the surrounding posterior nasal nerve against first distal portion (142) of guide catheter (114). In some versions, such compression of the inferior turbinate (IT) and the surrounding posterior nasal nerve may be sufficient to crush the posterior nasal nerve without fracturing the inferior turbinate (IT). In some instances, such crushing of the posterior nasal nerve is sufficient to effectively disable the posterior nasal nerve, such that dilator (130) provides denervation.

Due to the general rigidity of guide catheter (114) and each guidewire (116, 118), in some versions the only forces exerted on the patient's anatomy by inflation of dilator (130) may be on the inferior turbinate (IT) and the surrounding posterior nasal nerve. In other words, the rigidity of first distal portion (142) of guide catheter (114) and/or first guidewire (116) may provide a mechanical ground that absorbs forces exerted by dilator (130) toward the nasal septum (NS), such that neither guide catheter (114) nor first guidewire (116) bears against the nasal septum (NS) during inflation of dilator (130) as dilator (130) compresses the inferior turbinate (IT) against guide catheter (114). In addition, or alternatively, the rigidity of second guidewire (118) may provide a mechanical ground that absorbs forces exerted by dilator (130) toward the lateral nasal wall (NW), such that neither dilator (130) nor second guidewire (118) bears against the lateral nasal wall (NW) during inflation of dilator (130). While at least a portion of ENT compression instrument (110) may incidentally contact the lateral nasal wall (NW) or the nasal septum (NS) during inflation of dilator (130), those anatomical structures are not moved or remodeled by inflation of dilator (130) in the present example. Thus, the forces and/or pressure exerted on the patient's anatomy by the inflation of dilator (130) may be asymmetric relative to a longitudinal axis of second guidewire (118) and/or shaft (122). By way of example, dilation catheter (112) may be constructed and operable in accordance with at least some of the teachings of U.S. Pat. No. 9,615,959, entitled “Uncinate Process Support for Ethmoid Infundibulum Illumination,” issued Apr. 11, 2017, the disclosure of which is incorporated by reference herein, in its entirety.

In the present example, during the stage shown in FIG. 13E, a lateral side of the expanded dilator (130) engages the lateral nasal wall (NW) such that the lateral nasal wall (NW) provides at least some degree of a mechanical ground for dilator (130) and thereby assists the expanded dilator (130) in applying pressure to the inferior turbinate (IT) medially. Nevertheless, the forces exerted by the lateral side of the expanded dilator (130) against the lateral nasal wall (NW) may be insufficient to substantially move or remodel the lateral nasal wall (NW). For example, such forces may be applied over a relatively large surface area contact such that the resulting pressure may be insufficient to move or remodel the lateral nasal wall (NW), at least by comparison to a relatively small surface area contact over which forces may be applied by the expanded dilator (130) against the inferior turbinate (IT). In addition, or alternatively, such forces may be exerted by a relatively conforming surface of the expanded dilator (130), at least by comparison to a relatively non-conforming surface of the expanded dilator (130) which may exert forces against the inferior turbinate (IT). In this regard, the side of the expanded dilator (130) which engages the inferior turbinate (IT) may have a substantially greater stiffness than that of the opposing side of the expanded dilator (130) which engages the lateral nasal wall (NW).

More particularly, dilator (130) may have an asymmetric configuration relative to the longitudinal axis of second guidewire (118) and/or shaft (122), at least when in the expanded state, for achieving an asymmetric application of forces and/or pressure on the patient's anatomy. For example, dilator (130) may include a plurality of protrusions, similar to secondary protrusions (58) described above in connection with FIG. 10, extending from a primary exterior surface of dilator (130) on a first (medial) side of shaft (122) and may lack such protrusions on a second (lateral) side of shaft (122). In such cases, dilator (130) may be configured to exert forces against the patient's anatomy on the first side of shaft (122) that are applied over relatively small surface area contact(s) via such protrusions, while dilator (130) may be configured to exert forces against the patient's anatomy on the second side of shaft (122) that are applied over a relatively large surface area contact via the primary exterior surface of dilator (130).

In some other versions, dilator (130) may have a triangular profile with rounded corners, similar to triangular profile (76) having rounded corners (78) described above in connection with FIG. 12C. In such cases, dilator (130) may be configured to exert forces against the patient's anatomy (e.g., the inferior turbinate (IT)) on a first side of shaft (122) that are applied over a relatively small surface area contact via one such rounded corner, while dilator (130) may be configured to exert forces against the patient's anatomy (e.g., the lateral nasal wall (NW)) on a second side of shaft (122) that are applied over a relatively large surface area contact via the opposing side of dilator (130). Various examples of asymmetric dilator configurations for achieving an asymmetric application of forces and/or pressure on the patient's anatomy to facilitate compression of the inferior turbinate (IT) and accompanying nasal nerve without remodeling the lateral nasal wall (NW) are described in greater detail below.

B. Exemplary ENT Compression Instrument with Articulatable Paddle

FIG. 14 shows a distal portion of another exemplary ENT compression instrument (210) that may be used to compress a posterior nasal nerve or some other anatomical structure (e.g., within the ear, nose, or throat, etc.). Compression instrument (210) of this example is similar to compression instrument (110) described above except as otherwise described below. In this regard, compression instrument (210) comprises a dilation catheter (212), a rigid, malleable, or steerable guide catheter (214), and a guidewire (218) slidably disposed within dilation catheter (212). Dilation catheter (212) includes an elongate shaft (222) having a distal end (224), with a dilator (230) positioned at or near distal end (224) of shaft (222). Shaft (222) further includes a first lumen (not shown) providing a pathway for fluid communication between an inflation fluid source and dilator (230), and a second lumen (not shown) that is configured to slidably receive guidewire (218). Guide catheter (214) also defines a lumen (not shown) extending therealong to an open distal end (248) that is configured to slidably receive guidewire (218) and dilation catheter (212), such that guide catheter (214) may guide each of guidewire (218) and dilator (230) out through open distal end (248). In some versions, guidewire (218) and the corresponding lumen of dilation catheter (212) are omitted.

Compression instrument (210) of the present example also includes an articulation member in the form of an articulatable paddle (250) extending outwardly (e.g., distally and/or laterally) from guide catheter (214) at or near open distal end (248). Paddle (250) may be formed of a substantially rigid material. In the example shown, paddle (250) is coupled to guide catheter (214) at or near open distal end (248) via an articulation joint (252) for facilitating articulation of paddle (250) relative to guide catheter (214). In this regard, articulation joint (252) may include an actuator or driver (not shown), such as a pull-wire and/or other structure, that is configured to selectively actuate articulation of paddle (250) relative to guide catheter (214). Such a driver may be in operative communication with a controller (not shown) for receiving control signals therefrom to initiate and/or arrest articulation of paddle (250). In some versions, paddle (250) is articulatable between a non-articulated state (not shown) in which paddle (250) extends generally parallel to a longitudinal axis of guide catheter (214) and at least one articulated state in which paddle (250) extends obliquely relative to the longitudinal axis of guide catheter (214). As shown, paddle (250) may also extend distally beyond guide catheter (214) when in the at least one articulated state such that open distal end (248) of guide catheter (214) is positioned proximally relative to a distal end of paddle (250). In this manner, open distal end (248) may be configured to face toward the inferior nasal meatus between the inferior turbinate (IT) and the lateral nasal wall (NW) while paddle (250) is positioned between the nasal septum (NS) and the inferior turbinate (IT), as shown in FIG. 14.

In an exemplary nasal nerve compression procedure, guide catheter (214) may initially be inserted into a nostril (not shown) of a patient and advanced distally through the nasal cavity to position paddle (250) between the nasal septum (NS) and the inferior turbinate (IT). In some versions, paddle (250) may initially be in the non-articulated state to assist with facilitating such initial insertion and may subsequently be articulated to an articulated state. In addition, or alternatively, paddle (250) may be selectively articulated between any number of articulated states during such initial insertion to assist with steering paddle (250) toward a suitable position between the nasal septum (NS) and the inferior turbinate (IT) for mechanically grounding the inferior turbinate (IT) during subsequent expansion of dilator (230). In some versions, paddle (250) includes one or more position sensors that is/are operable to generate signals indicating a real-time position of paddle (250) in three-dimensional space; and such signals are used to assist in positioning paddle (250) at the appropriate space between the nasal septum (NS) and the inferior turbinate (IT). In any event, open distal end (248) of guide catheter (214) may face toward the inferior nasal meatus between the inferior turbinate (IT) and the lateral nasal wall (NW) with paddle (250) positioned between the nasal septum (NS) and the inferior turbinate (IT), and guidewire (218) may be advanced distally through the lumen of guide catheter (214) and out of open distal end (248) of guide catheter (214) to position a distal portion of guidewire (218) in the inferior nasal meatus between the inferior turbinate (IT) and the lateral nasal wall (NW). Dilation catheter (212) may then be advanced distally through the lumen of guide catheter (214) along guidewire (218) and out of open distal end (248) of guide catheter (214) to position dilator (230) of dilation catheter (212) within the inferior nasal meatus between the inferior turbinate (IT) and the lateral nasal wall (NW) while dilator (230) is in the non-expanded state.

After dilator (230) has been positioned between the inferior turbinate (IT) and the lateral nasal wall (NW), dilator (230) may be expanded to the expanded state for applying pressure to the posterior nasal nerve surrounding the inferior turbinate (IT). More particularly, the expansion of dilator (230) urges the inferior turbinate (IT) medially, thereby compressing the inferior turbinate (IT) and the surrounding posterior nasal nerve against paddle (250). In some instances, such crushing of the posterior nasal nerve is sufficient to effectively disable the posterior nasal nerve, such that dilator (230) provides denervation. In some versions, paddle (250) may be articulated toward dilator (230) to further compress the inferior turbinate (IT) between paddle (250) and the expanded dilator (230).

C. Exemplary Dilation Catheter with Laterally-Opposed Balloons

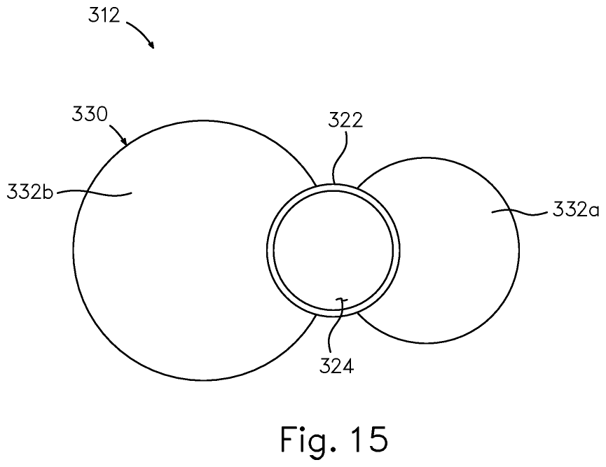

FIG. 15 shows a distal portion of another exemplary dilation catheter (312) for use with an ENT compression instrument (110, 210) that may be used to compress a posterior nasal nerve or some other anatomical structure (e.g., within the ear, nose, or throat, etc.). Dilation catheter (312) of this example is similar to dilation catheter (112) described above except as otherwise described below. In this regard, dilation catheter (312) includes an elongate shaft (322) having a distal end (324), with a dilator (330) positioned at or near distal end (324) of shaft (322). Shaft (322) further includes at least one lumen (not shown) providing a pathway for fluid communication between an inflation fluid source and dilator (330). In some versions, dilation catheter (312) includes one or more position sensors that is/are operable to generate signals indicating a real-time position of dilation catheter (312) in three-dimensional space. For instance, at least one position sensor may indicate the real-time position of distal end (324) in three-dimensional space. In addition, or in the alternative, at least one position sensor may indicate the real-time position of dilator (330) in three-dimensional space.

In the example shown, dilator (330) has an asymmetric configuration relative to a longitudinal axis of shaft (322), at least when in an expanded state, for achieving an asymmetric application of forces and/or pressure on the patient's anatomy. In this regard, dilator (330) of the present example comprises a pair of generally spherical inflatable balloons (332a, 332b), each having an interior that is in fluid communication with the inflation fluid source. The inflation fluid may thus be communicated from the inflation fluid source to each balloon (332a, 332b) to transition dilator (330) from a non-expanded state (not shown) to the illustrated expanded state; and back from each balloon (332a, 332b) to the inflation fluid source to transition dilator (330) from the illustrated expanded state back to the non-expanded state. In some versions, each balloon (332a, 332b) may be inflatable independently of the other balloon (332a, 332b). For example, shaft (322) may include a pair of lumens (not shown) isolated from each other for providing respective pathways for fluid communication between the inflation fluid source and an interior of a corresponding balloon (332a, 332b). In some versions, at least one balloon (332a, 332b) comprises an extensible material, such that dilator (330) is resiliently biased to assume the non-expanded state. In some other versions, at least one balloon (332a, 332b) comprises a flexible yet non-extensible material (e.g., mylar).

In any event, balloons (332a, 332b) are laterally opposed from each other relative to the longitudinal axis of shaft (322) such that each balloon (332a, 332b) extends laterally outwardly from a respective side of shaft (322) when inflated; and are configured differently from each other to provide differently-sized surface area contacts with the patient's anatomy. More particularly, first balloon (332a) has a relatively small cross dimension (e.g., diameter) when inflated, at least by comparison to a relatively large cross dimension (e.g., diameter) of second balloon (332b) when inflated, such that first balloon (332a) may provide a relatively small surface area contact with the patient's anatomy on a first side of shaft (322), at least by comparison to a relatively large surface area contact with the patient's anatomy provided by second balloon (332b) on a second side of shaft (322).

In this manner, dilator (330) may be configured to exert forces against the patient's anatomy on the first side of shaft (322) that are applied over the relatively small surface area contact via first balloon (332a), while dilator (330) may be configured to exert forces against the patient's anatomy on the second side of shaft (322) that are applied over the relatively large surface area contact via second balloon (332b). Thus, dilator (330) may be configured to exert locally concentrated forces against the patient's anatomy on the first side such that the resulting pressure may be sufficient to compress the patient's anatomy on the first side (e.g., the inferior turbinate (IT)) against a mechanical ground (e.g., provided by first distal portion (142) of instrument (110) or paddle (250) of instrument (210)), while dilator (330) may be configured to exert broadly distributed forces against the patient's anatomy on the second side such that the resulting pressure may be insufficient to move or remodel the patient's anatomy on the second side (e.g., the lateral nasal wall (NW)). For example, the pressure resulting from locally concentrated forces exerted against the inferior turbinate (IT) by dilator (330) may sufficiently crush the posterior nasal nerve to effectively disable the posterior nasal nerve, such that dilator (330) provides denervation.

While balloons (332a, 332b) have been described as being generally spherical, it will be appreciated that one or both balloons (332a, 332b) may alternatively have an elongate shape. For example, first balloon (332a) may be generally spherical while second balloon (332b) may be generally cylindrical to contribute to the asymmetric configuration of dilator (330) and, more particularly, to increase the surface area contact over which the forces exerted against the patient's anatomy on the second side are applied by second balloon (332b) relative to the surface area contact over which the forces exerted against the patient's anatomy on the first side are applied by first balloon (332a). As another example, both balloons (332a, 332b) may be generally cylindrical. In such cases, second balloon (332b) may have a relatively large length, at least by comparison to a relatively small length of first balloon (332a), to contribute to the asymmetric configuration of dilator (330) and, more particularly, to increase the surface area contact over which the forces exerted against the patient's anatomy on the second side are applied by second balloon (332b) relative to the surface area contact over which the forces exerted against the patient's anatomy on the first side are applied by first balloon (332a). In addition, or alternatively, first balloon (332a) may have a relatively high stiffness, at least by comparison to a relatively low stiffness of second balloon (332b), to promote compression of the patient's anatomy on the first side by first balloon (332a) and/or to inhibit movement or remodeling of the patient's anatomy on the second side by second balloon (332b). For example, second balloon (332b) may comprise an extensible material, while first balloon (332a) may comprise a non-extensible material. Thus, first balloon (332a) may be substantially non-conformable to the patient's anatomy on the first side of shaft (322), while second balloon (332b) may be substantially conformable to the patient's anatomy on the second side of shaft (322).

While dilator (330) has been described as comprising a laterally-opposed pair of balloons (332a, 332b), it will be appreciated that dilator (330) may comprise a laterally-opposed pair of any suitable expandable structures that are configured differently from each other to provide differently-sized surface area contacts with the patient's anatomy. For example, dilator (330) may comprise a laterally-opposed pair of generally spherical expandable baskets having different cross dimensions and/or stiffnesses from each other when expanded, in place of balloons (332a, 332b). Alternatively, dilator (330) may comprise a single generally spherical expandable basket in place of one of balloons (332a, 332b) and having a different cross dimension and/or stiffness from the other of balloons (332a, 332b).

D. Exemplary Dilation Catheter with Single Sided Balloon

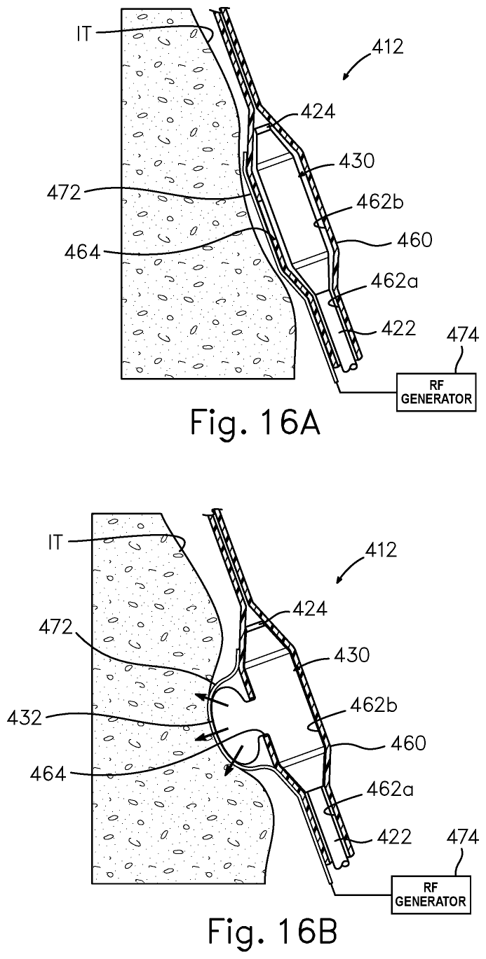

FIGS. 16A-16B show a distal portion of another exemplary dilation catheter (412) for use with an ENT compression instrument (110, 210) that may be used to compress a posterior nasal nerve or some other anatomical structure (e.g., within the ear, nose, or throat, etc.). Dilation catheter (412) of this example is similar to dilation catheter (112) described above except as otherwise described below. In this regard, dilation catheter (412) includes an elongate shaft (422) having a distal end (424), with a dilator (430) positioned at or near distal end (424) of shaft (422). Shaft (422) further includes a lumen (not shown) providing a pathway for fluid communication between an inflation fluid source and dilator (430). In some versions, dilation catheter (412) includes one or more position sensors that is/are operable to generate signals indicating a real-time position of dilation catheter (412) in three-dimensional space. For instance, at least one position sensor may indicate the real-time position of distal end (424) in three-dimensional space. In addition, or in the alternative, at least one position sensor may indicate the real-time position of dilator (430) in three-dimensional space.

In the example shown, dilator (430) has an asymmetric configuration relative to a longitudinal axis of shaft (422), at least when in an expanded state, for achieving an asymmetric application of forces and/or pressure on the patient's anatomy. In this regard, dilator (430) of the present example comprises a generally cylindrical inflatable balloon for selectively receiving inflation fluid from the inflation fluid source to transition dilator (430) between a non-expanded state (FIG. 16A) and an expanded state (FIG. 16B) in which a protruding portion (432) of dilator (430) extends laterally outwardly relative to a single side of shaft (422). To that end, dilator (430) is at least partially housed within an elongate rigid, malleable, and/or steerable sheath (460) which defines a hollow interior including a relatively narrow proximal portion (462a) in which shaft (422) is disposed, and a relatively wide distal portion (462b) in which dilator (430) is disposed. More particularly, a proximal portion of sheath (460) that is proximal to dilator (430) may be either steerable or malleable, while a distal portion of sheath (460) defining distal portion (462b) may be either rigid or resiliently biased to assume a contracted configuration. In some versions, at least one position sensor similar to those described above may indicate the real-time position of sheath (460) in three-dimensional space. As shown, a lateral bore (464) extends through a sidewall of sheath (460) on a first side of sheath (460) to distal portion (462b) of the hollow interior for permitting protruding portion (432) of dilator (430) to selectively extend laterally therethrough. In some versions, sheath (460) may have a sufficient degree of stiffness to resist deflection of sheath (460) during expansion of dilator (430) to an expanded state for applying pressure to the posterior nasal nerve surrounding the inferior turbinate (IT), as described in greater detail below.

In any event, distal portion (462b) of the hollow interior of sheath (460) is sized and shaped relative to dilator (430) to limit the expansion of dilator (430) therein, such that at least protruding portion (432) of dilator (430) may be forced to bulge out of the hollow interior through lateral bore (464) after reaching a threshold degree of expansion, as shown in FIG. 16B. In this manner, dilator (430) is generally constrained within distal portion (462b) of the hollow interior of sheath (460) when in the expanded state, except for protruding portion (432) which may extend laterally outwardly through lateral bore (464). Thus, dilator (430) is restricted by sheath (460) from extending laterally outwardly from a second side of sheath (460) opposite the first side on which lateral bore (464) is provided.

Dilation catheter (412) of the present example further comprises a generally linear wire (472) extending along an outer surface of sheath (460) over lateral bore (464). Wire (472) is sufficiently flexible to allow the selective extension of protruding portion (432) of dilator (430) through lateral bore (464) described above. During such extension, protruding portion (432) of dilator (430) may urge wire (472) from a non-deployed state (FIG. 16A) in which wire (472) is substantially flush with the outer surface of sheath (460), to a deployed state (FIG. 16B) in which wire (472) extends along a lateral surface of protruding portion (432) of dilator (430) and thereby protrudes laterally outwardly from the first side of sheath (460). In some versions, wire (472) may be formed of a resilient material (e.g., nitinol, etc.), such that wire (472) is resiliently biased to assume the non-deployed state of FIG. 16A. In any event, wire (472) may have a relatively small cross dimension (e.g., diameter), at least by comparison to a relatively large surface area of the lateral surface of protruding portion (432) of dilator (430), such that wire (472) may provide a relatively small surface area contact with the patient's anatomy on the first side of sheath (460), at least by comparison to a relatively large surface area contact which would otherwise be provided by protruding portion (432) of dilator (430) with the patient's anatomy on the first side of sheath (460) in the absence of wire (472). Thus, it will be appreciated that dilator (430) may exert forces against the patient's anatomy via wire (472) when dilator (430) and wire (472) are in the expanded and deployed states, respectively, such that wire (472) may serve to locally concentrate such forces against the patient's anatomy. In other versions, a pull-wire (not shown) may be secured to and extend proximally from a distal region of wire (472), such that the pull-wire may be selectively pulled proximally relative to sheath (460) to cause wire (472) to buckle outwardly and thereby transition from the non-deployed state to the deployed state for engaging the patient's anatomy. In such cases, dilator (430) may be omitted.

In this manner, dilator (430) and/or wire (472) may be configured to exert locally concentrated forces against the patient's anatomy on the first side of sheath (460) such that the resulting pressure may be sufficient to compress the patient's anatomy on the first side (e.g., the inferior turbinate (IT)) against a mechanical ground (e.g., provided by first distal portion (142) of instrument (110) or paddle (250) of instrument (210)), while the rigidity of sheath (460) may provide a mechanical ground that absorbs forces exerted by dilator (430) toward the patient's anatomy on the second side of sheath (460) (e.g., the lateral nasal wall (NW)), such that no forces are exerted against the patient's anatomy on the second side by dilation catheter (412).

In some other versions, a portion of the outer surface of sheath (460) on the second side may engage the patient's anatomy on the second side (e.g., the lateral nasal wall (NW)). In such cases, dilator (430) and/or wire (472) may be configured to exert forces against the patient's anatomy on the first side of sheath (460) that are applied over a relatively small surface area contact as described above, while such a portion of the outer surface of sheath (460) may be configured to exert forces against the patient's anatomy on the second side of sheath (460) that are applied over a relatively large surface area contact. Thus, dilator (430) and/or wire (472) may be configured to exert locally concentrated forces against the patient's anatomy on the first side such that the resulting pressure may be sufficient to compress the patient's anatomy on the first side (e.g., the inferior turbinate (IT)) against a mechanical ground (e.g., provided by first distal portion (142) of instrument (110) or paddle (250) of instrument (210)), while such a portion of the outer surface of sheath (460) may be configured to exert broadly distributed forces against the patient's anatomy on the second side such that the resulting pressure may be insufficient to move or remodel the patient's anatomy on the second side (e.g., the lateral nasal wall (NW)). For example, the pressure resulting from locally concentrated forces exerted against the inferior turbinate (IT) by dilator (430) may sufficiently crush the posterior nasal nerve to effectively disable the posterior nasal nerve, such that dilator (430) provides denervation.

In some versions, wire (472) may be electrically conductive and configured to deliver RF energy to tissue. For example, wire (472) may be electrically coupled with an RF generator (474). Wire (472) may thereby serve as an electrode operable to cooperate with a ground pad (not shown) placed in contact with the patient's skin to apply monopolar RF energy to tissue to ablate, electroporate, and/or cauterize the tissue, for example. In some such versions, an electrically insulating material (e.g., plastic, etc.) may be interposed between wire (472) and sheath (460), such that wire (472) may be electrically energized without also energizing sheath (460) or other portions of dilation catheter (412). In some other versions, a pair of wires (472) may be secured to sheath (460) and configured to deliver RF energy to tissue. For example, such wires (472) may each be electrically coupled with RF generator (474). The pair of wires (472) may thereby be operable to apply bipolar RF energy to tissue, with one wire (472) serving as an active electrode and the other wire (472) serving as a return electrode to ablate, electroporate, and/or cauterize the tissue, for example. In cases where wire(s) (472) applies either monopolar or bipolar RF energy to the inferior turbinate (IT), such RF energy may reach and sufficiently ablate the posterior nasal nerve to effectively disable the posterior nasal nerve and thereby provide denervation. Thus, dilation catheter (412) may provide mechanical denervation (e.g., via crushing of the nerve), RF denervation (e.g., via application of RF energy to the nerve), or a combination of mechanical and RF denervation.

E. Exemplary Dilation Catheter with Balloon and Helical Wire

FIG. 17 shows a distal portion of another exemplary dilation catheter (512) for use with an ENT compression instrument (110, 210) that may be used to compress a posterior nasal nerve or some other anatomical structure (e.g., within the ear, nose, or throat, etc.). Dilation catheter (512) of this example is similar to dilation catheter (112) described above except as otherwise described below. In this regard, dilation catheter (512) includes an elongate shaft (522) having a distal end (524), with a dilator (530) positioned at or near distal end (524) of shaft (522). Shaft (522) further includes a lumen (not shown) providing a pathway for fluid communication between an inflation fluid source and dilator (530). In some versions, dilation catheter (512) includes one or more position sensors that is/are operable to generate signals indicating a real-time position of dilation catheter (512) in three-dimensional space. For instance, at least one position sensor may indicate the real-time position of distal end (524) in three-dimensional space. In addition, or in the alternative, at least one position sensor may indicate the real-time position of dilator (530) in three-dimensional space.

In the example shown, dilator (530) has an asymmetric configuration relative to a longitudinal axis of shaft (522), at least when in an expanded state, for achieving an asymmetric application of forces and/or pressure on the patient's anatomy. In this regard, dilator (530) of the present example comprises a generally cylindrical inflatable balloon (532) and a generally helical wire (572) wrapped about a cylindrical outer surface of balloon (532) and configured to expand and contract with balloon (532). In particular, wire (572) is resiliently biased to contract to a non-expanded state (not shown); yet wire (572) is also flexible enough to expand with balloon (532) to achieve the illustrated expanded state, without substantially impeding the expansion of balloon (532). In some versions, wire (572) may be secured to balloon (532) via adhesive, for example. In some other versions, wire (572) may be slidably disposed in a helical sleeve (not shown) that is secured to an exterior of balloon (532). In such cases, wire (572) may be proximally retracted relative to balloon (532) when balloon (532) is in a non-expanded state and may be distally advanced over balloon (532) when balloon (532) is in the expanded state, such that wire (572) does not necessarily need to be configured to expand and contract with balloon (532). In any event, wire (572) may have a relatively small cross dimension (e.g., diameter), at least by comparison to a relatively large surface area of the underlying cylindrical outer surface of balloon (532), such that wire (572) may provide a relatively small surface area contact with the patient's anatomy, at least by comparison to a relatively large surface area contact which would otherwise be provided by balloon (532) with the patient's anatomy. Thus, it will be appreciated that dilator (530) may exert forces against the patient's anatomy via wire (572) when dilator (530) is in the expanded state, such that wire (572) may serve to locally concentrate such forces against the patient's anatomy.

In this manner, dilator (530) may be configured to exert forces against the patient's anatomy on the first side of shaft (522) that are applied over the relatively small surface area contact via wire (572). Thus, dilator (530) may be configured to exert locally concentrated forces against the patient's anatomy on the first side such that the resulting pressure may be sufficient to compress the patient's anatomy on the first side (e.g., the inferior turbinate (IT)) against a mechanical ground (e.g., provided by first distal portion (142) of instrument (110) or paddle (250) of instrument (210)). In some cases, the presence of such a mechanical ground positioned between the inferior turbinate (IT) and the nasal septum (NS) may assist with inhibiting dilator (530) from applying sufficient pressure to the lateral nasal wall (NW) to move or remodel the lateral nasal wall (NW). In any event, the pressure resulting from locally concentrated forces exerted against the inferior turbinate (IT) by dilator (530) may sufficiently crush the posterior nasal nerve to effectively disable the posterior nasal nerve, such that dilator (530) provides denervation.

In addition, or alternatively, wire (572) may have a relatively high stiffness, at least by comparison to a relatively low stiffness of balloon (532), to promote compression of the patient's anatomy on the first side by dilator (530) and/or to inhibit movement or remodeling of the patient's anatomy on the second side by dilator (530). For example, wire (572) may be substantially non-conformable to the patient's anatomy, while balloon (532) may be substantially conformable to the patient's anatomy. In some versions, wire (572) may be electrically conductive and configured to deliver RF energy to tissue. For example, wire (572) may be routed along shaft (522) and electrically coupled with an RF generator (574). Wire (572) may thereby serve as an electrode operable to cooperate with a ground pad (not shown) placed in contact with the patient's skin to apply monopolar RF energy to tissue to ablate, electroporate, and/or cauterize the tissue, for example. In some such versions, an electrically insulating material (e.g., plastic, etc.) may be interposed between wire (572) and shaft (522), such that wire (572) may be electrically energized without also energizing shaft (522) or other portions of dilation catheter (512). In cases where wire (572) applies monopolar RF energy to the inferior turbinate (IT), such RF energy may reach and sufficiently ablate the posterior nasal nerve to effectively disable the posterior nasal nerve and thereby provide denervation. Thus, dilation catheter (512) may provide mechanical denervation (e.g., via crushing of the nerve), RF denervation (e.g., via application of RF energy to the nerve), or a combination of mechanical and RF denervation.

In addition to the foregoing, at least part of dilator (530) and/or any other component of dilation catheter (512) may be configured and operable in accordance with at least some of the teachings of U.S. Pat. No. 8,961,495, entitled “Devices, Systems and Methods for Treating Disorders of the Ear, Nose, and Throat,” issued Feb. 24, 2015, the disclosure of which is incorporated by reference herein, in its entirety; and/or U.S. Pat. No. 10,485,609, entitled “Dilation Balloon with RF Energy Delivery Feature,” issued Nov. 26, 2019, the disclosure of which is incorporated by reference herein, in its entirety; and/or U.S. Pub. No. 2022/0087739, entitled “ENT Instrument with Expandable Ablation Feature,” published Mar. 24, 2022, the disclosure of which is incorporated by reference herein, in its entirety.

F. Exemplary Dilation Catheter with Balloon and Linear Wires

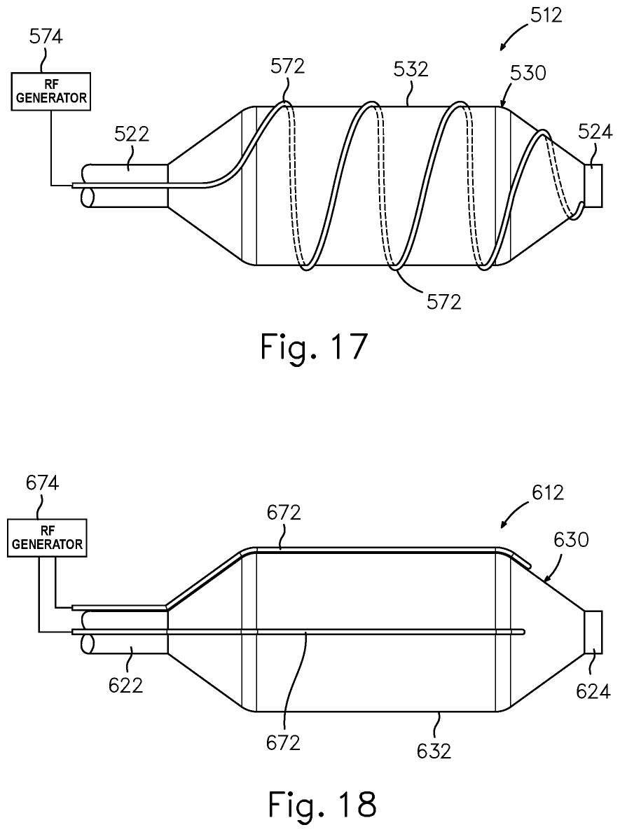

FIG. 18 shows a distal portion of another exemplary dilation catheter (612) for use with an ENT compression instrument (110, 210) that may be used to compress a posterior nasal nerve or some other anatomical structure (e.g., within the ear, nose, or throat, etc.). Dilation catheter (612) of this example is similar to dilation catheter (112) described above except as otherwise described below. In this regard, dilation catheter (612) includes an elongate shaft (622) having a distal end (624), with a dilator (630) positioned at or near distal end (624) of shaft (622). Shaft (622) further includes a lumen (not shown) providing a pathway for fluid communication between an inflation fluid source and dilator (630). In some versions, dilation catheter (612) includes one or more position sensors that is/are operable to generate signals indicating a real-time position of dilation catheter (612) in three-dimensional space. For instance, at least one position sensor may indicate the real-time position of distal end (624) in three-dimensional space. In addition, or in the alternative, at least one position sensor may indicate the real-time position of dilator (630) in three-dimensional space.

In the example shown, dilator (630) has an asymmetric configuration relative to a longitudinal axis of shaft (622), at least when in an expanded state, for achieving an asymmetric application of forces and/or pressure on the patient's anatomy. In this regard, dilator (630) of the present example comprises a generally cylindrical inflatable balloon (632) and a pair of generally linear wires (672) extending along a semicylindrical outer surface of balloon (632) and configured to collectively expand and contract with balloon (632). In particular, wires (672) are each resiliently biased to contract to a non-expanded state (not shown); yet wires (672) are also each flexible enough to expand with balloon (632) to achieve the illustrated expanded state, without substantially impeding the expansion of balloon (632). In some versions, wires (672) may each be secured to balloon (632) via adhesive, for example. In any event, wires (672) may each have a relatively small cross dimension (e.g., diameter), at least by comparison to a relatively large surface area of the underlying semicylindrical outer surface of balloon (632), such that wires (672) may each provide a relatively small surface area contact with the patient's anatomy, at least by comparison to a relatively large surface area contact which would otherwise be provided by balloon (632) with the patient's anatomy. Thus, it will be appreciated that dilator (630) may exert forces against the patient's anatomy via wires (672) when dilator (630) is in the expanded state, such that wires (672) may serve to locally concentrate such forces against the patient's anatomy.

More particularly, wires (672) each extend along a semicylindrical surface of balloon (632) on a first side of shaft (622) and the opposing semicylindrical outer surface of balloon (632) is left exposed on a second side of shaft (622), such that one or both wires (672) may engage the patient's anatomy on the first side of shaft (622) without engaging the patient's anatomy on the second side of shaft (622). For example, the patient's anatomy on the second side of shaft (622) may instead be engaged directly by balloon (632), such that balloon (632) may provide a relatively large surface area contact with the patient's anatomy on the second side of shaft (622), at least by comparison to the relatively small surface area contact(s) with the patient's anatomy provided by one or both wires (672) on the first side of shaft (622).

In this manner, dilator (630) may be configured to exert forces against the patient's anatomy on the first side of shaft (622) that are applied over the relatively small surface area contact(s) via one or both wires (672), while dilator (630) may be configured to exert forces against the patient's anatomy on the second side of shaft (622) that are applied over the relatively large surface area contact via balloon (632). Thus, dilator (630) may be configured to exert locally concentrated forces against the patient's anatomy on the first side such that the resulting pressure may be sufficient to compress the patient's anatomy on the first side (e.g., the inferior turbinate (IT)) against a mechanical ground (e.g., provided by first distal portion (142) of instrument (110) or paddle (250) of instrument (210)), while dilator (630) may be configured to exert broadly distributed forces against the patient's anatomy on the second side such that the resulting pressure may be insufficient to move or remodel the patient's anatomy on the second side (e.g., the lateral nasal wall (NW)). For example, the pressure resulting from locally concentrated forces exerted against the inferior turbinate (IT) by dilator (630) may sufficiently crush the posterior nasal nerve to effectively disable the posterior nasal nerve, such that dilator (630) provides denervation.

In addition, or alternatively, wires (672) may each have a relatively high stiffness, at least by comparison to a relatively low stiffness of balloon (632), to promote compression of the patient's anatomy on the first side by dilator (630) and/or to inhibit movement or remodeling of the patient's anatomy on the second side by dilator (630). For example, wire (672) may be substantially non-conformable to the patient's anatomy on the first side of shaft (622), while balloon (632) may be substantially conformable to the patient's anatomy on the second side of shaft (622).

In some versions, one or both wires (672) may be electrically conductive and configured to deliver RF energy to tissue. For example, wires (672) may each be routed along shaft (622) and electrically coupled with an RF generator (674). Wires (672) may thereby each serve as an electrode operable to cooperate with a ground pad (not shown) placed in contact with the patient's skin to apply monopolar RF energy to tissue to ablate, electroporate, and/or cauterize the tissue, for example. In some such versions, an electrically insulating material (e.g., plastic, etc.) may be interposed between each wire (672) and shaft (622), such that wires (672) may be electrically energized without also energizing shaft (622) or other portions of dilation catheter (612). In some other versions, wires (672) may be operable to apply bipolar RF energy to tissue, with one wire (672) serving as an active electrode and the other wire (672) serving as a return electrode to ablate, electroporate, and/or cauterize the tissue, for example. In cases where wires (672) apply either monopolar or bipolar RF energy to the inferior turbinate (IT), such RF energy may reach and sufficiently ablate the posterior nasal nerve to effectively disable the posterior nasal nerve and thereby provide denervation. Thus, dilation catheter (612) may provide mechanical denervation (e.g., via crushing of the nerve), RF denervation (e.g., via application of RF energy to the nerve), or a combination of mechanical and RF denervation.

In addition to the foregoing, at least part of dilator (630) and/or any other component of dilation catheter (612) may be configured and operable in accordance with at least some of the teachings of U.S. Pat. No. 8,961,495, entitled “Devices, Systems and Methods for Treating Disorders of the Ear, Nose, and Throat,” issued Feb. 24, 2015, the disclosure of which is incorporated by reference herein, in its entirety; and/or U.S. Pat. No. 10,485,609, entitled “Dilation Balloon with RF Energy Delivery Feature,” issued Nov. 26, 2019, the disclosure of which is incorporated by reference herein, in its entirety; and/or U.S. Pub. No. 2022/0087739, entitled “ENT Instrument with Expandable Ablation Feature,” published Mar. 24, 2022, the disclosure of which is incorporated by reference herein, in its entirety.

G. Exemplary Dilation Catheter with Spherical Balloon and Hemispherical Expandable Basket

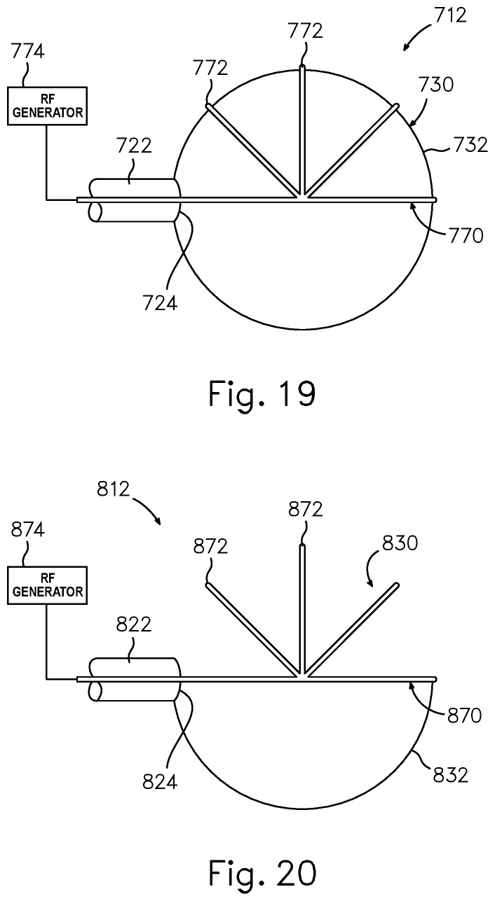

FIG. 19 shows a distal portion of another exemplary dilation catheter (712) for use with an ENT compression instrument (110, 210) that may be used to compress a posterior nasal nerve or some other anatomical structure (e.g., within the ear, nose, or throat, etc.). Dilation catheter (712) of this example is similar to dilation catheter (112) described above except as otherwise described below. In this regard, dilation catheter (712) includes an elongate shaft (722) having a distal end (724), with a dilator (730) positioned at or near distal end (724) of shaft (722). Shaft (722) further includes a lumen (not shown) providing a pathway for fluid communication between an inflation fluid source and dilator (730). In some versions, dilation catheter (712) includes one or more position sensors that is/are operable to generate signals indicating a real-time position of dilation catheter (712) in three-dimensional space. For instance, at least one position sensor may indicate the real-time position of distal end (724) in three-dimensional space. In addition, or in the alternative, at least one position sensor may indicate the real-time position of dilator (730) in three-dimensional space.

In the example shown, dilator (730) has an asymmetric configuration relative to a longitudinal axis of shaft (722), at least when in an expanded state, for achieving an asymmetric application of forces and/or pressure on the patient's anatomy. In this regard, dilator (730) of the present example comprises a generally spherical inflatable balloon (732) and a generally hemispherical expandable basket (770) extending over a hemispherical outer surface of balloon (732) and configured to expand and contract with balloon (732). In particular, basket (770) is resiliently biased to contract to a non-expanded state (not shown); yet basket (770) is also flexible enough to expand with balloon (732) to achieve the illustrated expanded state, without substantially impeding the expansion of balloon (732). In some versions, basket (770) may be secured to balloon (732) via adhesive, for example, at one or more discrete, predetermined locations selected to facilitate coordinated expansion of basket (770) with balloon (732). In any event, basket (770) includes a plurality of arched elongate members in the form of strips or beams (772), which may each have a generally rectangular cross section with a relatively small width, at least by comparison to a relatively large surface area of the underlying hemispherical outer surface of balloon (732), such that beams (772) may each provide a relatively small surface area contact with the patient's anatomy, at least by comparison to a relatively large surface area contact which would otherwise be provided by balloon (732) with the patient's anatomy. Thus, it will be appreciated that dilator (730) may exert forces against the patient's anatomy via one or more beams (772) of basket (770) when dilator (730) is in the expanded state, such that the one or more beams (772) of basket (770) may serve to locally concentrate such forces against the patient's anatomy. While elongate members in the form of beams (772) having generally rectangular cross sections are described herein, it will be appreciated that elongate members having any suitable cross-sectional configuration may be used in place of beams (772). For example, elongate members in the form of wires having generally circular cross sections may be used in place of beams (772). In such cases, each wire may have a relatively small diameter, at least by comparison to a relatively large surface area of the underlying hemispherical outer surface of balloon (732). In other versions, each beam (772) may have a generally triangular cross section, with a flat base of each beam (772) facing balloon (730) and an opposing peak of each beam (772) facing outwardly to engage the patient's anatomy.

More particularly, basket (770) extends over a hemispherical outer surface of balloon (732) on a first side of shaft (722) and the opposing hemispherical outer surface of balloon (732) is left exposed on a second side of shaft (722), such that basket (770) may engage the patient's anatomy on the first side of shaft (722) without engaging the patient's anatomy on the second side of shaft (722). For example, the patient's anatomy on the second side of shaft (722) may instead be engaged directly by the opposing hemispherical outer surface of balloon (732), such that balloon (732) may provide a relatively large surface area contact with the patient's anatomy on the second side of shaft (722), at least by comparison to the relatively small surface area contact(s) with the patient's anatomy provided by one or more beams (772) of basket (770) on the first side of shaft (722).

In this manner, dilator (730) may be configured to exert forces against the patient's anatomy on the first side of shaft (722) that are applied over the relatively small surface area contact(s) via one or more beams (772) of basket (770), while dilator (730) may be configured to exert forces against the patient's anatomy on the second side of shaft (722) that are applied over the relatively large surface area contact via balloon (732). Thus, dilator (730) may be configured to exert locally concentrated forces against the patient's anatomy on the first side such that the resulting pressure may be sufficient to compress the patient's anatomy on the first side (e.g., the inferior turbinate (IT)) against a mechanical ground (e.g., provided by first distal portion (142) of instrument (110) or paddle (250) of instrument (210)), while dilator (730) may be configured to exert broadly distributed forces against the patient's anatomy on the second side such that the resulting pressure may be insufficient to move or remodel the patient's anatomy on the second side (e.g., the lateral nasal wall (NW)). For example, the pressure resulting from locally concentrated forces exerted against the inferior turbinate (IT) by dilator (730) may sufficiently crush the posterior nasal nerve to effectively disable the posterior nasal nerve, such that dilator (730) provides denervation.

In some other versions, protruding portions of balloon (732) may be forced to bulge outwardly between adjacent pairs of beams (772) after reaching a threshold degree of expansion. Such protruding portions of balloon (732) may be configured to apply substantially uniform pressure against the patient's anatomy on the first side.

In addition, or alternatively, beams (772) may each have a relatively high stiffness, at least by comparison to a relatively low stiffness of balloon (732), to promote compression of the patient's anatomy on the first side by dilator (730) and/or to inhibit movement or remodeling of the patient's anatomy on the second side by dilator (730). For example, beams (772) may each be substantially non-conformable to the patient's anatomy on the first side of shaft (722), while balloon (732) may be substantially conformable to the patient's anatomy on the second side of shaft (722).

In some versions, basket (770) may be electrically conductive and configured to deliver RF energy to tissue. For example, at least one beam (772) of basket (770) may be routed along shaft (722) and electrically coupled with an RF generator (774). Basket (770) may thereby serve as an electrode operable to cooperate with a ground pad (not shown) placed in contact with the patient's skin to apply monopolar RF energy to tissue to ablate, electroporate, and/or cauterize the tissue, for example. In some such versions, an electrically insulating material (e.g., plastic, etc.) may be interposed between basket (770) and shaft (722), such that basket (770) may be electrically energized without also energizing shaft (722) or other portions of dilation catheter (712). In cases where basket (770) applies monopolar RF energy to the inferior turbinate (IT), such RF energy may reach and sufficiently ablate the posterior nasal nerve to effectively disable the posterior nasal nerve and thereby provide denervation. Thus, dilation catheter (712) may provide mechanical denervation (e.g., via crushing of the nerve), RF denervation (e.g., via application of RF energy to the nerve), or a combination of mechanical and RF denervation.

In addition to the foregoing, at least part of dilator (730) and/or any other component of dilation catheter (712) may be configured and operable in accordance with at least some of the teachings of U.S. Pat. No. 7,654,997, entitled “Devices, Systems and Methods for Diagnosing and Treating Sinusitus and Other Disorders of the Ears, Nose and/or Throat,” issued Feb. 2, 2010, the disclosure of which is incorporated by reference herein, in its entirety; U.S. Pat. No. 8,961,495, entitled “Devices, Systems and Methods for Treating Disorders of the Ear, Nose, and Throat,” issued Feb. 24, 2015, the disclosure of which is incorporated by reference herein, in its entirety; and/or U.S. Pat. No. 10,485,609, entitled “Dilation Balloon with RF Energy Delivery Feature,” issued Nov. 26, 2019, the disclosure of which is incorporated by reference herein, in its entirety; and/or U.S. Pub. No. 2022/0087739, entitled “ENT Instrument with Expandable Ablation Feature,” published Mar. 24, 2022, the disclosure of which is incorporated by reference herein, in its entirety.

H. Exemplary Dilation Catheter with Hemispherical Balloon and Hemispherical Expandable Basket

FIG. 20 shows a distal portion of another exemplary dilation catheter (812) for use with an ENT compression instrument (110, 210) that may be used to compress a posterior nasal nerve or some other anatomical structure (e.g., within the ear, nose, or throat, etc.). Dilation catheter (812) of this example is similar to dilation catheter (112) described above except as otherwise described below. In this regard, dilation catheter (812) includes an elongate shaft (822) having a distal end (824), with a dilator (830) positioned at or near distal end (824) of shaft (822). Shaft (822) further includes a lumen (not shown) providing a pathway for fluid communication between an inflation fluid source and dilator (830). In some versions, dilation catheter (812) includes one or more position sensors that is/are operable to generate signals indicating a real-time position of dilation catheter (812) in three-dimensional space. For instance, at least one position sensor may indicate the real-time position of distal end (824) in three-dimensional space. In addition, or in the alternative, at least one position sensor may indicate the real-time position of dilator (830) in three-dimensional space.

In the example shown, dilator (830) has an asymmetric configuration relative to a longitudinal axis of shaft (822), at least when in an expanded state, for achieving an asymmetric application of forces and/or pressure on the patient's anatomy. In this regard, dilator (830) of the present example comprises a generally hemispherical inflatable balloon (832) and a generally hemispherical expandable basket (870) aligned with balloon (832) to collectively provide dilator (830) with a generally spherical shape. Basket (870) is configured to expand and contract either with or independently of balloon (832). In some versions, basket (870) may be resiliently biased to contract to a non-expanded state (not shown); yet basket (870) may also be flexible enough to expand with balloon (832) to achieve the illustrated expanded state, without substantially impeding the expansion of balloon (832). In such cases, basket (770) may be secured to balloon (732) via adhesive, for example, at one or more discrete, predetermined locations selected to facilitate coordinated expansion of basket (870) with balloon (832). In other versions, basket (870) may be resiliently biased to expand to the illustrated expanded state; yet basket (870) may also be flexible enough to contract within a sheath (not shown) in which dilator (830) may be slidably and/or coaxially disposed. In such cases, the sheath may be proximally retractable relative to dilator (830) to facilitate expansion of basket (870) independently of balloon (832).

In any event, basket (870) includes a plurality of arched elongate members in the form of strips or beams (872), which may each have a generally rectangular cross section with a relatively small width, at least by comparison to a relatively large surface area of the outer surface of balloon (832), such that beams (872) may each provide a relatively small surface area contact with the patient's anatomy, at least by comparison to a relatively large surface area contact provided by balloon (832) with the patient's anatomy. Thus, it will be appreciated that dilator (830) may exert forces against the patient's anatomy via one or more beams (872) of basket (870) when dilator (830) is in the expanded state, such that the one or more beams (872) of basket (870) may serve to locally concentrate such forces against the patient's anatomy. While elongate members in the form of beams (872) having generally rectangular cross sections are described herein, it will be appreciated that elongate members having any suitable cross-sectional configuration may be used in place of beams (872). For example, elongate members in the form of wires having generally circular cross sections may be used in place of beams (872). In such cases, each wire may have a relatively small diameter, at least by comparison to a relatively large surface area of the outer surface of balloon (832). In other versions, each beam (872) may have a generally triangular cross section, with a peak of each beam (872) facing outwardly to engage the patient's anatomy.

More particularly, basket (870) is positioned on a first side of shaft (822) and the hemispherical outer surface of balloon (832) is left exposed on a second side of shaft (822), such that basket (870) may engage the patient's anatomy on the first side of shaft (822) without engaging the patient's anatomy on a second side of shaft (822). For example, the patient's anatomy on the second side of shaft (822) may instead be engaged by balloon (832), such that balloon (832) may provide a relatively large surface area contact with the patient's anatomy on the second side of shaft (822), at least by comparison to the relatively small surface area contact(s) with the patient's anatomy provided by one or more beams (872) of basket (870) on the first side of shaft (822).

In this manner, dilator (830) may be configured to exert forces against the patient's anatomy on the first side of shaft (822) that are applied over the relatively small surface area contact(s) via one or more beams (872) of basket (870), while dilator (830) may be configured to exert forces against the patient's anatomy on the second side of shaft (822) that are applied over the relatively large surface area contact via balloon (832). Thus, dilator (830) may be configured to exert locally concentrated forces against the patient's anatomy on the first side such that the resulting pressure may be sufficient to compress the patient's anatomy on the first side (e.g., the inferior turbinate (IT)) against a mechanical ground (e.g., provided by first distal portion (142) of instrument (110) or paddle (250) of instrument (210)), while dilator (830) may be configured to exert broadly distributed forces against the patient's anatomy on the second side such that the resulting pressure may be insufficient to move or remodel the patient's anatomy on the second side (e.g., the lateral nasal wall (NW)). For example, the pressure resulting from locally concentrated forces exerted against the inferior turbinate (IT) by dilator (830) may sufficiently crush the posterior nasal nerve to effectively disable the posterior nasal nerve, such that dilator (830) provides denervation.

In addition, or alternatively, beams (872) may each have a relatively high stiffness, at least by comparison to a relatively low stiffness of balloon (832), to promote compression of the patient's anatomy on the first side by dilator (830) and/or to inhibit movement or remodeling of the patient's anatomy on the second side by dilator (830). For example, beams (872) may each be substantially non-conformable to the patient's anatomy on the first side of shaft (822), while balloon (832) may be substantially conformable to the patient's anatomy on the second side of shaft (822).

In some versions, basket (870) may be electrically conductive and configured to deliver RF energy to tissue. For example, at least one beam (872) of basket (870) may be routed along shaft (822) and electrically coupled with an RF generator (874). Basket (870) may thereby serve as an electrode operable to cooperate with a ground pad (not shown) placed in contact with the patient's skin to apply monopolar RF energy to tissue to ablate, electroporate, and/or cauterize the tissue, for example. In some such versions, an electrically insulating material (e.g., plastic, etc.) may be interposed between basket (870) and shaft (822), such that basket (870) may be electrically energized without also energizing shaft (822) or other portions of dilation catheter (812). In cases where basket (870) applies monopolar RF energy to the inferior turbinate (IT), such RF energy may reach and sufficiently ablate the posterior nasal nerve to effectively disable the posterior nasal nerve and thereby provide denervation. Thus, dilation catheter (812) may provide mechanical denervation (e.g., via crushing of the nerve), RF denervation (e.g., via application of RF energy to the nerve), or a combination of mechanical and RF denervation.

In addition to the foregoing, at least part of dilator (830) and/or any other component of dilation catheter (812) may be configured and operable in accordance with at least some of the teachings of U.S. Pat. No. 7,654,997, entitled “Devices, Systems and Methods for Diagnosing and Treating Sinusitus and Other Disorders of the Ears, Nose and/or Throat,” issued Feb. 2, 2010, the disclosure of which is incorporated by reference herein, in its entirety; U.S. Pat. No. 8,961,495, entitled “Devices, Systems and Methods for Treating Disorders of the Ear, Nose, and Throat,” issued Feb. 24, 2015, the disclosure of which is incorporated by reference herein, in its entirety; and/or U.S. Pat. No. 10,485,609, entitled “Dilation Balloon with RF Energy Delivery Feature,” issued Nov. 26, 2019, the disclosure of which is incorporated by reference herein, in its entirety; and/or U.S. Pub. No. 2022/0087739, entitled “ENT Instrument with Expandable Ablation Feature,” published Mar. 24, 2022, the disclosure of which is incorporated by reference herein, in its entirety.

I. Exemplary Dilation Catheter with Spherical Expandable Basket Having Pair of Hemispherical Portions

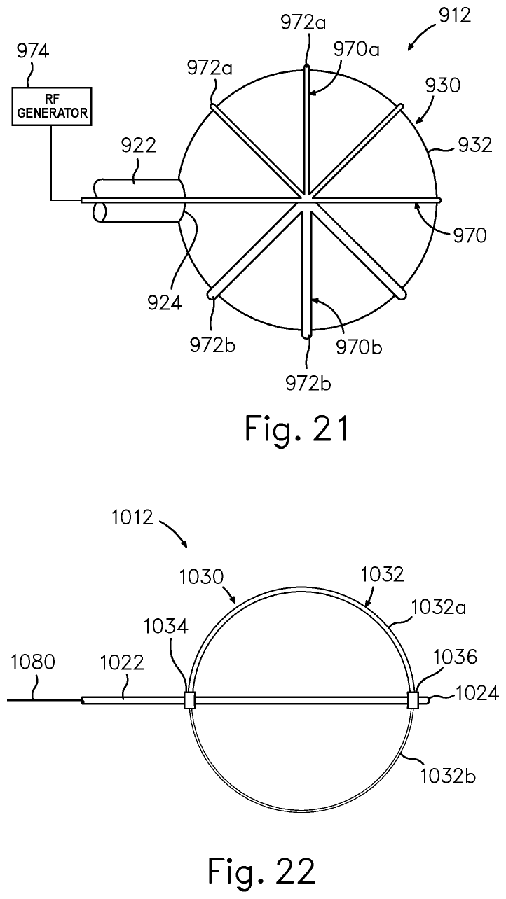

FIG. 21 shows a distal portion of another exemplary dilation catheter (912) for use with an ENT compression instrument (110, 210) that may be used to compress a posterior nasal nerve or some other anatomical structure (e.g., within the ear, nose, or throat, etc.). Dilation catheter (912) of this example is similar to dilation catheter (112) described above except as otherwise described below. In this regard, dilation catheter (912) includes an elongate shaft (922) having a distal end (924), with a dilator (930) positioned at or near distal end (924) of shaft (922). Shaft (922) further includes a lumen (not shown) providing a pathway for fluid communication between an inflation fluid source and dilator (930). In some versions, dilation catheter (912) includes one or more position sensors that is/are operable to generate signals indicating a real-time position of dilation catheter (912) in three-dimensional space. For instance, at least one position sensor may indicate the real-time position of distal end (924) in three-dimensional space. In addition, or in the alternative, at least one position sensor may indicate the real-time position of dilator (930) in three-dimensional space.

In the example shown, dilator (930) has an asymmetric configuration relative to a longitudinal axis of shaft (922), at least when in an expanded state, for achieving an asymmetric application of forces and/or pressure on the patient's anatomy. In this regard, dilator (930) of the present example comprises a generally spherical inflatable balloon (932) and a generally spherical expandable basket (970) having first and second hemispherical basket portions (970a, 970b) extending over respective hemispherical outer surfaces of balloon (932) and configured to expand and contract with balloon (932). In particular, basket (970) is resiliently biased to contract to a non-expanded state (not shown); yet basket (970) is also flexible enough to expand with balloon (932) to achieve the illustrated expanded state, without substantially impeding the expansion of balloon (932). In some versions, basket (970) may be secured to balloon (932) via adhesive, for example, at one or more discrete, predetermined locations selected to facilitate coordinated expansion of basket (970) with balloon (932). In other versions, balloon (932) may be omitted, and basket (970) may be resiliently biased to expand to the illustrated expanded state; yet basket (970) may also be flexible enough to contract within a sheath (not shown) in which dilator (930) may be slidably and/or coaxially disposed. In such cases, the sheath may be proximally retractable relative to dilator (930) to facilitate expansion of basket (970) in the absence of balloon (932).

In any event, each basket portion (970a, 970b) of basket (970) includes a respective plurality of arched elongate members in the form of beams (972a, 972b). More particularly, first basket portion (970a) includes a plurality of first arched beams (972a), which may each have a generally rectangular cross section with a relatively small width, at least by comparison to a relatively large width of the generally rectangular cross section of each second arched beam (972b) of second basket portion (970b), such that first beams (972a) may each provide a relatively small surface area contact with the patient's anatomy, at least by comparison to a relatively large surface area contact provided by each second beam (972b) with the patient's anatomy. Thus, it will be appreciated that dilator (930) may exert forces against the patient's anatomy via one or more first beams (972a) of first basket portion (970a) when dilator (930) is in the expanded state, such that the one or more first beams (972) of first basket portion (970a) may serve to locally concentrate such forces against the patient's anatomy, at least to a greater degree than any local concentration of forces provided by second beams (972b) of second basket portion (970b). While elongate members in the form of beams (972a, 972b) having generally rectangular cross sections are described herein, it will be appreciated that elongate members having any suitable cross-sectional configuration may be used in place of beams (972a, 972b). For example, elongate members in the form of wires having generally circular cross sections may be used in place of beams (972a, 972b). In such cases, each wire of first basket portion (970a) may have a relatively small diameter, at least by comparison to a relatively large diameter of each wire of second basket portion (970b). In other versions, each first beam (972a) may have a generally triangular cross section, with a flat base of each first beam (972a) facing balloon (930) and an opposing peak of each first beam (972a) facing outwardly to engage the patient's anatomy.

More particularly, first basket portion (970a) extends over a hemispherical outer surface of balloon (932) on a first side of shaft (922) and second basket portion (970b) extends over the opposing hemispherical outer surface of balloon (932) on a second side of shaft (922), such that first basket portion (970a) may engage the patient's anatomy on the first side of shaft (922) without engaging the patient's anatomy on the second side of shaft (922). For example, the patient's anatomy on the second side of shaft (922) may instead be engaged by second basket portion (970b), such that one or more second beams (972b) of second basket portion (970b) may provide a relatively large surface area contact with the patient's anatomy on the second side of shaft (922), at least by comparison to the relatively small surface area contact(s) with the patient's anatomy provided by one or more first beams (972a) of first basket portion (970a) on the first side of shaft (922).

In this manner, dilator (930) may be configured to exert forces against the patient's anatomy on the first side of shaft (922) that are applied over the relatively small surface area contact(s) via one or more first beams (972a) of first basket portion (970a), while dilator (930) may be configured to exert forces against the patient's anatomy on the second side of shaft (922) that are applied over the relatively large surface area contact(s) via one or more second beams (972b) of second basket portion (970b). Thus, dilator (930) may be configured to exert locally concentrated forces against the patient's anatomy on the first side such that the resulting pressure may be sufficient to compress the patient's anatomy on the first side (e.g., the inferior turbinate (IT)) against a mechanical ground (e.g., provided by first distal portion (142) of instrument (110) or paddle (250) of instrument (210)), while dilator (930) may be configured to exert broadly distributed forces against the patient's anatomy on the second side such that the resulting pressure may be insufficient to move or remodel the patient's anatomy on the second side (e.g., the lateral nasal wall (NW)). For example, the pressure resulting from locally concentrated forces exerted against the inferior turbinate (IT) by dilator (930) may sufficiently crush the posterior nasal nerve to effectively disable the posterior nasal nerve, such that dilator (930) provides denervation.

In some other versions, protruding portions of balloon (932) may be forced to bulge outwardly between adjacent pairs of beams (972a, 972b) after reaching a threshold degree of expansion. Such protruding portions of balloon (932) may be configured to apply substantially uniform pressure against the patient's anatomy on the respective first or second side.

In addition, or alternatively, first beams (972a) may each have a relatively high stiffness, at least by comparison to a relatively low stiffness of second beams (972b), to promote compression of the patient's anatomy on the first side by dilator (930) and/or to inhibit movement or remodeling of the patient's anatomy on the second side by dilator (930). For example, first beams (972a) may each be substantially non-conformable to the patient's anatomy on the first side of shaft (922), while second beams (972b) may each be substantially conformable to the patient's anatomy on the second side of shaft (922).

In some versions, a proximal end of basket (970) may be fixedly secured to distal end (924) of shaft (922), and a pull-wire (not shown) may be secured to and extend proximally from a distal end of basket (970), such that the pull-wire may be selectively pulled proximally relative to shaft (922) when basket (970) is in the expanded state to retract the distal end of basket (970) proximally toward the proximal end of basket (970). For example, the distal end of basket (970) may be retracted proximally toward the proximal end of basket (970) while shaft (922) holds the proximal end of basket (970) stationary, thereby causing basket (970) to buckle outwardly such that first basket portion (970a) may provide localized pressure against the patient's anatomy on the first side (e.g., the inferior turbinate (IT)), while second basket portion (970b) may conformably collapse against the patient's anatomy on the second side (e.g., the lateral nasal wall (NW)) to provide grounding support without substantially affecting the patient's anatomy on the second side.

In some versions, first basket portion (970a) may be electrically conductive and configured to deliver RF energy to tissue. For example, at least one first beam (972a) of first basket portion (970a) may be routed along shaft (922) and electrically coupled with an RF generator (974). First basket portion (970a) may thereby serve as an electrode operable to cooperate with a ground pad (not shown) placed in contact with the patient's skin to apply monopolar RF energy to tissue to ablate, electroporate, and/or cauterize the tissue, for example. In some such versions, an electrically insulating material (e.g., plastic, etc.) may be interposed between first basket portion (970a) and each of second basket portion (970b) and shaft (922), such that first basket portion (970a) may be electrically energized without also energizing second basket portion (970b), shaft (922) or other portions of dilation catheter (912). In cases where first basket portion (970a) applies monopolar RF energy to the inferior turbinate (IT), such RF energy may reach and sufficiently ablate the posterior nasal nerve to effectively disable the posterior nasal nerve and thereby provide denervation. Thus, dilation catheter (412) may provide mechanical denervation (e.g., via crushing of the nerve), RF denervation (e.g., via application of RF energy to the nerve), or a combination of mechanical and RF denervation.

In addition to the foregoing, at least part of dilator (930) and/or any other component of dilation catheter (912) may be configured and operable in accordance with at least some of the teachings of U.S. Pat. No. 7,654,997, entitled “Devices, Systems and Methods for Diagnosing and Treating Sinusitus and Other Disorders of the Ears, Nose and/or Throat,” issued Feb. 2, 2010, the disclosure of which is incorporated by reference herein, in its entirety; U.S. Pat. No. 8,961,495, entitled “Devices, Systems and Methods for Treating Disorders of the Ear, Nose, and Throat,” issued Feb. 24, 2015, the disclosure of which is incorporated by reference herein, in its entirety; and/or U.S. Pat. No. 10,485,609, entitled “Dilation Balloon with RF Energy Delivery Feature,” issued Nov. 26, 2019, the disclosure of which is incorporated by reference herein, in its entirety; and/or U.S. Pub. No. 2022/0087739, entitled “ENT Instrument with Expandable Ablation Feature,” published Mar. 24, 2022, the disclosure of which is incorporated by reference herein, in its entirety.

J. Exemplary Dilation Catheter with Spherical Balloon Having Pair of Hemispherical Portions

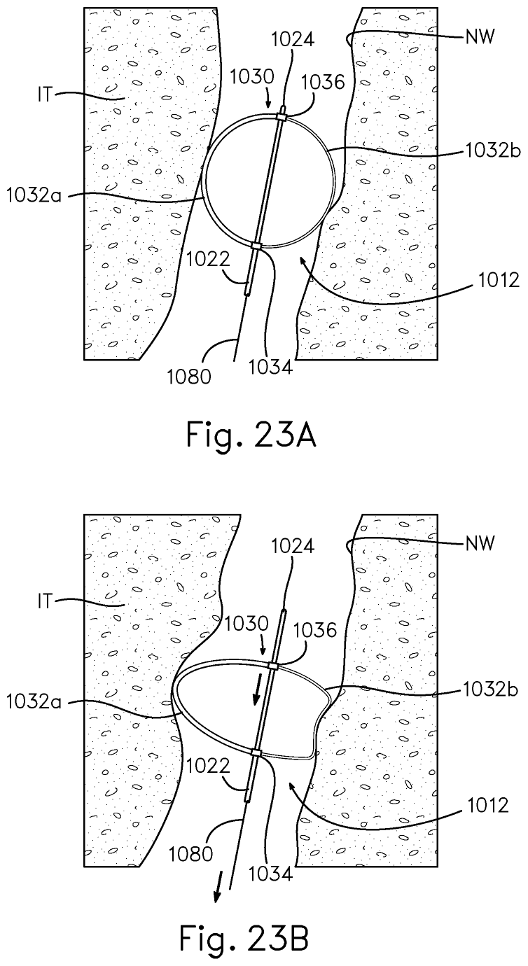

FIGS. 22-23B show a distal portion of another exemplary dilation catheter (1012) for use with an ENT compression instrument (110, 210) that may be used to compress a posterior nasal nerve or some other anatomical structure (e.g., within the ear, nose, or throat, etc.). Dilation catheter (1012) of this example is similar to dilation catheter (112) described above except as otherwise described below. In this regard, dilation catheter (1012) includes an elongate shaft (1022) having a distal end (1024), with a dilator (1030) positioned at or near distal end (1024) of shaft (1022). Shaft (1022) further includes at least one lumen (not shown) providing a pathway for fluid communication between an inflation fluid source and dilator (1030). In some versions, dilation catheter (1012) includes one or more position sensors that is/are operable to generate signals indicating a real-time position of dilation catheter (1012) in three-dimensional space. For instance, at least one position sensor may indicate the real-time position of distal end (1024) in three-dimensional space. In addition, or in the alternative, at least one position sensor may indicate the real-time position of dilator (1030) in three-dimensional space.

In the example shown, dilator (1030) has an asymmetric configuration relative to a longitudinal axis of shaft (1022), at least when in an expanded state, for achieving an asymmetric application of forces and/or pressure on the patient's anatomy. In this regard, dilator (1030) of the present example comprises a generally spherical inflatable balloon (1032) having first and second hemispherical balloon portions (1032a, 1032b) each having an interior that is in fluid communication with the inflation fluid source. The inflation fluid may thus be communicated from the inflation fluid source to each balloon portion (1032a, 1032b) to transition dilator (1030) from a non-expanded state (not shown) to an expanded state (FIG. 23A); and back from each balloon portion (1032a, 1032b) to the inflation fluid source to transition dilator (1030) from the expanded state (FIG. 23A) back to the non-expanded state. In some versions, each balloon portion (1032a, 1032b) may be inflatable independently of the other balloon portion (1032a, 1032b). For example, shaft (1022) may include a pair of lumens (not shown) isolated from each other for providing respective pathways for fluid communication between the inflation fluid source and an interior of a corresponding balloon portion (1032a, 1032b). In such cases, the interiors of balloon portions (1032a, 1032b) may likewise be isolated from each other. In some versions, at least one balloon portion (1032a, 1032b) comprises an extensible material, such that dilator (1030) is resiliently biased to assume the non-expanded state. In some other versions, at least one balloon portion (1032a, 1032b) comprises a flexible yet non-extensible material (e.g., mylar).

In the example shown, dilator (1030) includes a proximal coupling (1034) which fixedly secures a proximal end of balloon (1032) to shaft (1022) at a position proximal of distal end (1024) of shaft (1022), and a distal coupling (1036) which slidably secures a distal end of balloon (1032) to shaft (1022). In other versions, the proximal end of balloon (1032) may be fixedly secured to shaft (1022) at distal end (1024) of shaft (1022), such that balloon (1032) extends distally from shaft (1022) to position the distal end of balloon (1032) distal of distal end (1024) of shaft (1022). In either case, distal coupling (1036) may be retractable together with the distal end of balloon (1032) toward proximal coupling (1034) and the proximal end of balloon (1032) (e.g., along the longitudinal axis of shaft (1022)), as described in greater detail below.

In any event, balloon portions (1032a, 1032b) are laterally opposed from each other relative to the longitudinal axis of shaft (1022) such that each balloon portion (1032a, 1032b) extends laterally outwardly from a respective side of shaft (1022) when inflated, and are configured differently from each other to provide differently-sized surface area contacts with the patient's anatomy. More particularly, first balloon portion (1032a) has a relatively large material thickness, at least by comparison to a relatively small material thickness of second balloon portion (1032b), such that first balloon portion (1032a) may have a relatively high stiffness, at least by comparison to a relatively low stiffness of second balloon portion (1032b). Thus, first balloon portion (1032a) may be substantially non-conformable to the patient's anatomy on a first side of shaft (1022), while second balloon portion (1032b) may be substantially conformable to the patient's anatomy on a second side of shaft (1022), such that first balloon portion (1032a) may provide a relatively small surface area contact with the patient's anatomy on the first side of shaft (1022), at least by comparison to a relatively large surface area contact with the patient's anatomy provided by second balloon portion (1032b) on a second side of shaft (1022).

Dilation catheter (1012) of the present example further comprises a pull-wire (1080) secured to and extending proximally from distal coupling (1036) of dilator (1030). In this regard, shaft (1022) may include another lumen (not shown) that is configured to slidably receive pull-wire (1080) for routing pull-wire (1080) to a proximal end of dilation catheter (1012). In any event, pull-wire (1080) may be selectively pulled proximally relative to shaft (1022) when balloon (1032) is in the expanded state to retract the distal end of balloon (1032) proximally toward the proximal end of balloon (1032) and thereby transition dilator (1030) from a non-actuated, expanded state (FIG. 23A) to an actuated, expanded state (FIG. 23B). As shown in FIG. 23A, dilator (1030) may have a generally spherical shape described above when in the non-actuated, expanded state. Due to the retraction of the distal end of balloon (1032) toward the proximal end of balloon (1032) and the relative stiffnesses of balloon portions (1032a, 1032b), first balloon portion (1032a) may bow laterally outwardly without conforming to the patient's anatomy on the first side of shaft (1022) and thereby provide the relatively small surface area contact therewith, while second balloon portion (1032b) may deform laterally inwardly or otherwise conform to the patient's anatomy on the second side of shaft (1022) and thereby provide the relatively large surface area contact therewith when dilator (1030) is in the actuated, expanded state, as shown in FIG. 23B.

In this manner, dilator (1030) may be configured to exert forces against the patient's anatomy on the first side of shaft (1022) that are applied over the relatively small surface area contact via first balloon portion (1032a), while dilator (1030) may be configured to exert forces against the patient's anatomy on the second side of shaft (1022) that are applied over the relatively large surface area contact via second balloon portion (1032b). Thus, dilator (1030) may be configured to exert locally concentrated forces against the patient's anatomy on the first side such that the resulting pressure may be sufficient to compress the patient's anatomy on the first side (e.g., the inferior turbinate (IT)) against a mechanical ground (e.g., provided by first distal portion (142) of instrument (110) or paddle (250) of instrument (210)), while dilator (1030) may be configured to exert broadly distributed forces against the patient's anatomy on the second side such that the resulting pressure may be insufficient to move or remodel the patient's anatomy on the second side (e.g., the lateral nasal wall (NW)). For example, the pressure resulting from locally concentrated forces exerted against the inferior turbinate (IT) by dilator (1030) may sufficiently crush the posterior nasal nerve to effectively disable the posterior nasal nerve, such that dilator (1030) provides denervation.

In some versions, dilator (1030) may further include one or more elongate members in the form of strips/beams or wires extending along an outer surface of balloon (1032), such as along an outer surface of first balloon portion (1032a), such that dilator (1030) may exert forces against the patient's anatomy via such elongate members when dilator (1030) is in the expanded state to further locally concentrate such forces against the patient's anatomy. While dilator (1030) has been described as comprising a balloon (1032) having a laterally-opposed pair of balloon portions (1032a, 1032b), it will be appreciated that dilator (1030) may comprise a laterally-opposed pair of any suitable expandable structures that are configured differently from each other to provide differently-sized surface area contacts with the patient's anatomy, such as any of those described above.

In some versions, dilator (1030) may include one or more RF electrodes secured to an outer surface of balloon (1032), such as to the outer surface of first balloon portion (1032a), for delivering RF energy to tissue. For example, a single electrode may be electrically coupled with an RF generator (not shown) and may thereby be operable to cooperate with a ground pad (not shown) placed in contact with the patient's skin to apply monopolar RF energy to tissue to ablate, electroporate, and/or cauterize the tissue, for example. In some other versions, a pair of electrodes including an active electrode and a return electrode may be electrically coupled with the RF generator and may thereby be operable to apply bipolar RF energy to tissue to ablate, electroporate, and/or cauterize the tissue, for example. In cases where one or more electrodes apply either monopolar or bipolar RF energy to the inferior turbinate (IT), such RF energy may reach and sufficiently ablate the posterior nasal nerve to effectively disable the posterior nasal nerve and thereby provide denervation. Thus, dilation catheter (1012) may provide mechanical denervation (e.g., via crushing of the nerve), RF denervation (e.g., via application of RF energy to the nerve), or a combination of mechanical and RF denervation.

### V. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A method comprising: (a) inserting a first dilation catheter into a first nostril of a patient; (b) positioning a first dilator of the first dilation catheter between a nasal septum of the patient and a turbinate of the patient; (c) expanding the first dilator, thereby remodeling two or more of the nasal septum, the turbinate, or mucosal tissue of the patient; and (d) removing the first dilation catheter from the first nostril of the patient.

### Example 2

The method of Example 1, wherein the nasal septum is deviated before the act of inserting the first dilation catheter, wherein the nasal septum is substantially straightened after the act of removing the first dilation catheter.

### Example 3

The method of any one or more of Examples 1 through 2, wherein the first dilator comprises a balloon, wherein the act of expanding the first dilator comprises communicating an inflation fluid to the balloon.

### Example 4

The method of any one or more of Examples 1 through 3, wherein the act of expanding the first dilator comprises medializing the nasal septum.

### Example 5

The method of any one or more of Examples 1 through 4, wherein the act of expanding the first dilator comprises lateralizing the turbinate.

### Example 6

The method of any one or more of Examples 1 through 5, wherein the turbinate comprises an inferior turbinate.

### Example 7

The method of any one or more of Examples 1 through 6, wherein the expanded first dilator comprises a friction enhancing feature.

### Example 8

The method of any one or more of Examples 1 through 7, wherein the act of remodeling two or more of the nasal septum, the turbinate, or mucosal tissue of the patient comprises fracturing one or both of bone or cartilage in the nasal septum.

### Example 9

The method of any one or more of Examples 1 through 8, wherein the act of remodeling two or more of the nasal septum, the turbinate, or mucosal tissue of the patient comprises fracturing bone in the turbinate.

### Example 10

The method of any one or more of Examples 1 through 9, further comprising: (a) inserting a second dilation catheter into a second nostril of the patient; (b) positioning a second dilator of the second dilation catheter adjacent to the nasal septum of the patient; (c) expanding the second dilator; and (d) removing the second dilator from the second nostril of the patient.

### Example 11

The method of Example 10, wherein the act of positioning the second dilator comprises positioning the second dilator at a depth corresponding to a depth of the positioned first dilator, such that the first and second dilators are at corresponding depths on opposite sides of the nasal septum.

### Example 12

The method of any one or more of Examples 10 through 11, wherein the first and second dilators are expanded simultaneously.

### Example 13

The method of Example 12, wherein the first and second dilators exert opposing medial forces on the nasal septum.

### Example 14

The method of Example 13, wherein the expanded first dilator urges the nasal septum medially from a deviated configuration toward a substantially straight configuration, wherein the expanded second dilator prevents over-medialization of the nasal septum by the expanded first dilator.

### Example 15

The method of any one or more of Examples 10 through 14, wherein the first and second dilators are inflatable, wherein the act of expanding the first dilator comprises communicating inflation fluid from an inflation fluid source to the first dilator, wherein the act of expanding the second dilator comprises communicating inflation fluid from the inflation fluid source to the second dilator.

### Example 16

A method comprising: (a) positioning a first dilator adjacent to a first side of a nasal septum in a nasal cavity of a patient; (b) positioning a second dilator adjacent to a second side of the nasal septum; (c) expanding the positioned first dilator; and (d) expanding the positioned second dilator; wherein the expansion of the positioned first dilator urges the nasal septum toward the second dilator, wherein the expansion of the positioned second dilator restricts movement of the urged nasal septum.

### Example 17

The method of Example 16, wherein the nasal septum is deviated laterally from a central plane before the acts of positioning the first and second dilators, wherein the expanded first dilator urges the nasal septum medially toward the central plane.

### Example 18

The method of Example 17, wherein the expanded second dilator prevents movement of the medialized nasal septum past the central plane.

### Example 19

A method comprising: (a) inserting a first dilation catheter into a first nostril of a patient; (b) positioning a first dilator of the first dilation catheter between a first side of a deviated nasal septum of the patient and a turbinate of the patient; (c) inserting a second dilation catheter into a second nostril of the patient; (d) positioning a second dilator of the second dilation catheter adjacent to a second side of the deviated nasal septum of the patient; (e) expanding the positioned first dilator to medialize the deviated nasal septum and thereby remodeling the deviated nasal to achieve a substantially straight configuration of the nasal septum; and (f) expanding the positioned second dilator to restrict movement of the nasal septum beyond the substantially straight configuration.

### Example 20

The method of Example 19, wherein expanding the positioned first dilator further lateralizes the turbinate of the patient and thereby remodels the turbinate of the patient.

### Example 21

A method comprising: (a) inserting a dilation catheter into a nostril of a patient; (b) positioning a first dilator of the dilation catheter between a turbinate of the patient and an adjacent lateral nasal wall of the patient; (c) expanding the first dilator, thereby applying pressure to the turbinate of the patient; and (d) removing the dilation catheter from the nostril of the patient.

### Example 22

The method of Example 21, further comprising positioning a mechanical grounding member between the turbinate of the patient and a nasal septum of the patient, wherein the act of expanding the first dilator includes compressing the turbinate of the patient against the mechanical grounding member.

### Example 23

The method of Example 22, wherein the mechanical grounding member includes a second dilator.

### Example 24

The method of Example 22, wherein the mechanical grounding member includes a guide catheter.

### Example 25

The method of Example 22, wherein the mechanical grounding member includes a paddle.

### Example 26

The method of any of Examples 21 through 25, wherein the act of expanding the first dilator includes applying pressure to a nasal nerve within, extending through, or surrounding the turbinate.

### Example 27

The method of any of Examples 21 through 26, wherein the turbinate comprises an inferior turbinate.

### Example 28

The method of any of Examples 21 through 27, wherein the act of expanding the first dilator includes asymmetrically expanding the first dilator relative to a longitudinal axis of the dilation catheter.

### Example 29

The method of Example 28, wherein the first dilator is housed within a sheath, wherein the act of asymmetrically expanding the first dilator includes extending a protruding portion of the first dilator laterally outwardly through a lateral bore of the sheath.

### Example 30

The method of Example 28, wherein the first dilator includes first and second expandable portions laterally opposed from each other, wherein the act of asymmetrically expanding the first dilator includes expanding the first dilator to an expanded state.

### Example 31

The method of Example 30, wherein the first expandable portion has a first cross dimension when in the expanded state, wherein the second expandable portion has a second cross dimension different from the first cross dimension when in the expanded state.

### Example 32

The method of any of Examples 30 through 31, wherein the first expandable portion has a first stiffness when in the expanded state, wherein the second expandable portion has a second stiffness different from the first stiffness when in the expanded state.

### Example 33

The method of any of Examples 21 through 32, wherein the act of expanding the first dilator includes applying pressure to the turbinate of the patient via at least one wire.

### Example 34

The method of Example 33, further comprising applying RF energy to the turbinate via the at least one wire.

### Example 35

The method of any of Examples 21 through 34, further comprising retracting a distal end of the first dilator toward a proximal end of the first dilator.

### Example 36

A method comprising: (a) positioning a first dilator adjacent to a first side of a turbinate in a nasal cavity of a patient; (b) positioning a mechanical grounding member adjacent to a second side of the turbinate; and (c) expanding the positioned first dilator, wherein the expansion of the positioned first dilator compresses the turbinate against the mechanical grounding member.

### Example 37

The method of Example 36, wherein the expansion of the positioned first dilator applies pressure to a nasal nerve within, extending through, or surrounding the turbinate.

### Example 38

The method of any of Examples 36 through 37, wherein the mechanical grounding member includes at least one of a second dilator, a guide catheter, or a paddle.

### Example 39

A method comprising: (a) inserting a dilation catheter into a nostril of a patient; (b) positioning a dilator of the dilation catheter between a turbinate of the patient and an adjacent lateral nasal wall of the patient; and (c) expanding the positioned dilator to apply a first pressure to the turbinate and a second pressure to the lateral nasal wall, wherein the first pressure is greater than the second pressure.

### Example 40

The method of Example 39, wherein the first pressure is applied over a first surface area contact between the dilator and the turbinate, wherein the second pressure is applied over a second surface area contact between the dilator and the lateral nasal wall, wherein the first surface area contact is smaller than the second surface area contact.

### Example 41

An apparatus, comprising: (a) a dilation catheter configured to be inserted into a cavity of a patient's head, the dilation catheter comprising: (i) a shaft having a distal end, the shaft defining a longitudinal axis, and (ii) a first dilator at or near the distal end of the shaft, the first dilator being expandable from a non-expanded state to an expanded state; and (b) a mechanical grounding member, wherein the first dilator and the mechanical grounding member are configured to cooperate with each other to compress a turbinate of the patient when the first dilator is in the expanded state.

### Example 42

The apparatus of Example 41, further comprising a guide catheter configured to direct the dilation catheter into the cavity of the patient's head.

### Example 43

The apparatus of Example 42, wherein the mechanical grounding member is articulatable relative to the guide catheter.

### Example 44

The apparatus of any of Examples 41 through 43, wherein the mechanical grounding member includes a paddle.

### Example 45

The apparatus of any of Examples 41 through 43, wherein the mechanical grounding member includes a second dilator.

### Example 46

The apparatus of Example 42, wherein the guide catheter is bifurcated, wherein the mechanical grounding member includes a distal portion of the guide catheter.

### Example 47

The apparatus of any of Examples 41 through 46, wherein the first dilator has an asymmetric configuration relative to the longitudinal axis when in the expanded state.

### Example 48

The apparatus of Example 47, wherein the first dilator comprises: (A) an inflatable balloon having an interior cavity configured to receive an inflation fluid for expanding the first dilator from the non-expanded state to the expanded state, and (B) at least one wire positioned on the inflatable balloon.

### Example 49

The apparatus of Example 48, wherein the at least one wire is at least one of linear or helical.

### Example 50

The apparatus of any of Examples 48 through 49, wherein the at least one wire is operatively coupled with an RF energy source for delivering RF energy to the turbinate via the at least one wire.

### Example 51

The apparatus of any of Example 47, further comprising a sheath having a lateral bore, wherein the first dilator is housed within the sheath, wherein a protruding portion of the first dilator is configured to extend laterally outwardly through the lateral bore when in the expanded state.

### Example 52

The apparatus of Example 47, wherein the first dilator includes first and second expandable portions laterally opposed from each other.

### Example 53

The apparatus of Example 52, wherein the first expandable portion has a first cross dimension when in the expanded state, wherein the second expandable portion has a second cross dimension different from the first cross dimension when in the expanded state.

### Example 54

The apparatus of any of Examples 52 through 53, wherein the first expandable portion has a first stiffness when in the expanded state, wherein the second expandable portion has a second stiffness different from the first stiffness when in the expanded state.

### Example 55

The apparatus of any of Examples 52 through 54, wherein at least one of the first or second expandable portions includes at least one of an inflatable balloon or an expandable basket.

### Example 56

An apparatus, comprising: (a) a first guidewire configured to be inserted into a first cavity of a patient's head; (b) a guide catheter having a first lumen configured to receive the first guidewire; (c) a dilation catheter comprising: (i) a shaft configured to be received within the first lumen, the shaft having a second lumen configured to be advanced along the first guidewire, the shaft defining a longitudinal axis, and (ii) a dilator coupled to the shaft for advancement therewith along the first guidewire to position the dilator in the first cavity of the patient's head, the dilator being expandable from a non-expanded state to an expanded state; and (d) a mechanical grounding member configured to be inserted into a second cavity of the patient's head when the dilator is positioned in the first cavity of the patient's head, wherein the dilator and the mechanical grounding member are configured to cooperate with each other to compress a turbinate of the patient when the dilator is in the expanded state.

### Example 57

The apparatus of Example 56, further comprising a second guidewire configured to be inserted into the second cavity of the patient's head, wherein the guide catheter has a third lumen configured to be advanced along the second guidewire to position the mechanical grounding member in the second cavity of the patient's head.

### Example 58

The apparatus of any of Examples 56 through 57, wherein the mechanical grounding member is articulatable relative to the guide catheter.

### Example 59

The apparatus of any of Examples 56 through 58, wherein the dilator has an asymmetric configuration relative to the longitudinal axis when in the expanded state.

### Example 60

An apparatus, comprising: (a) a bifurcated guide catheter having first and second distal portions, the first distal portion being configured to be inserted into a first cavity of a patient's head; and (b) a dilation catheter comprising: (i) a shaft configured to be advanced into a second cavity of the patient's head through the second distal portion of the bifurcated guide catheter when the first distal portion is positioned in the first cavity of the patient's head, and (ii) a dilator coupled to the shaft for advancement therewith through the second distal portion of the bifurcated guide catheter to position the dilator in the second cavity of the patient's head, the dilator being expandable from a non-expanded state to an expanded state, wherein the dilator and the first distal portion of the bifurcated guide catheter are configured to cooperate with each other to compress a turbinate of the patient when the dilator is in the expanded state.

### VI. Miscellaneous

It should be understood that any of the examples described herein may include various other features in addition to or in lieu of those described above. By way of example only, any of the examples described herein may also include one or more of the various features disclosed in any of the various references that are incorporated by reference herein.

It should be understood that any one or more of the teachings, expressions, embodiments, examples, etc. described herein may be combined with any one or more of the other teachings, expressions, embodiments, examples, etc. that are described herein. The above-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Versions of the devices disclosed herein can be designed to be disposed of after a single use, or they can be designed to be used multiple times. Versions may, in either or both cases, be reconditioned for reuse after at least one use. Reconditioning may include any combination of the steps of disassembly of the device, followed by cleaning or replacement of particular pieces, and subsequent reassembly. In particular, versions of the device may be disassembled, and any number of the particular pieces or parts of the device may be selectively replaced or removed in any combination. Upon cleaning and/or replacement of particular parts, versions of the device may be reassembled for subsequent use either at a reconditioning facility, or by a surgical team immediately prior to a surgical procedure. Those skilled in the art will appreciate that reconditioning of a device may utilize a variety of techniques for disassembly, cleaning/replacement, and reassembly. Use of such techniques, and the resulting reconditioned device, are all within the scope of the present application.

By way of example only, versions described herein may be processed before surgery. First, a new or used instrument may be obtained and if necessary cleaned. In some instances, the instrument may be placed in a reprocessing tray (e.g., a metal bin or basket) and then cleaned in a surgical instrument washer. The instrument may then be sterilized. In one sterilization technique, the instrument is placed in a closed and sealed container, such as a plastic or TYVEK bag. The container and instrument may then be placed in a field of radiation that can penetrate the container, such as gamma radiation, x-rays, or high-energy electrons. The radiation may kill bacteria on the instrument and in the container. The sterilized instrument may then be stored in the sterile container. The sealed container may keep the instrument sterile until it is opened in a surgical facility. A device may also be sterilized using any other technique known in the art, including but not limited to beta or gamma radiation, ethylene oxide, steam, hydrogen peroxide vapor (e.g., via a STERRAD sterilization system by Advanced Sterilization Products of Irvine, Calif.), and/or using any other suitable systems or techniques.

Having shown and described various versions of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, versions, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

I/We claim:

1. A method comprising:
(a) inserting a dilation catheter into a nostril of a patient;
(b) positioning a first dilator of the dilation catheter between a turbinate of the patient and an adjacent lateral nasal wall of the patient;
(c) expanding the first dilator, thereby applying pressure to the turbinate of the patient; and
(d) removing the dilation catheter from the nostril of the patient.

2. The method of claim 1, further comprising positioning a mechanical grounding member between the turbinate of the patient and a nasal septum of the patient, wherein the act of expanding the first dilator includes compressing the turbinate of the patient against the mechanical grounding member.

3. The method of claim 2, wherein the mechanical grounding member includes a second dilator.

4. The method of claim 2, wherein the mechanical grounding member includes a guide catheter.

5. The method of claim 2, wherein the mechanical grounding member includes a paddle.

6. The method of claim 1, wherein the act of expanding the first dilator includes applying pressure to a nasal nerve within, extending through, or surrounding the turbinate.

7. The method of claim 1, wherein the turbinate comprises an inferior turbinate.

8. The method of claim 1, wherein the act of expanding the first dilator includes asymmetrically expanding the first dilator relative to a longitudinal axis of the dilation catheter.

9. The method of claim 8, wherein the first dilator is housed within a sheath, wherein the act of asymmetrically expanding the first dilator includes extending a protruding portion of the first dilator laterally outwardly through a lateral bore of the sheath.

10. The method of claim 8, wherein the first dilator includes first and second expandable portions laterally opposed from each other, wherein the act of asymmetrically expanding the first dilator includes expanding the first dilator to an expanded state.

11. The method of claim 10, wherein the first expandable portion has a first cross dimension when in the expanded state, wherein the second expandable portion has a second cross dimension different from the first cross dimension when in the expanded state.

12. The method of claim 10, wherein the first expandable portion has a first stiffness when in the expanded state, wherein the second expandable portion has a second stiffness different from the first stiffness when in the expanded state.

13. The method of claim 1, wherein the act of expanding the first dilator includes applying pressure to the turbinate of the patient via at least one wire.

14. The method of claim 13, further comprising applying RF energy to the turbinate via the at least one wire.

15. The method of claim 1, further comprising retracting a distal end of the first dilator toward a proximal end of the first dilator.

16. A method comprising:
(a) positioning a first dilator adjacent to a first side of a turbinate in a nasal cavity of a patient;
(b) positioning a mechanical grounding member adjacent to a second side of the turbinate; and
(c) expanding the positioned first dilator,
wherein the expansion of the positioned first dilator compresses the turbinate against the mechanical grounding member.

17. The method of claim 16, wherein the expansion of the positioned first dilator applies pressure to a nasal nerve within, extending through, or surrounding the turbinate.

18. The method of any of claim 16, wherein the mechanical grounding member includes at least one of a second dilator, a guide catheter, or a paddle.

19. An apparatus, comprising:
(a) a dilation catheter configured to be inserted into a cavity of a patient's head, the dilation catheter comprising:
(i) a shaft having a distal end, the shaft defining a longitudinal axis, and
(ii) a first dilator at or near the distal end of the shaft, the first dilator being expandable from a non-expanded state to an expanded state; and
(b) a mechanical grounding member,
wherein the first dilator and the mechanical grounding member are configured to cooperate with each other to compress a turbinate of the patient when the first dilator is in the expanded state.

20. The apparatus of claim 19, wherein the mechanical grounding member includes a second dilator.
